(19) United States
(12) Patent Application Publication
(10) Pub. No.: US 2024/0245804 A1
(43) Pub. Date: Jul. 25, 2024

(54) Expression of Bacterial Dinucleotide Cyclases

(71) Applicants: McMaster University, Hamilton (CA); Ottawa Hospital Research Institute, Ottawa (CA)

(72) Inventors: Brian Lichty, Brantford (CA); Fuan Wang, London (CA); Matthew Atherton, Pittsburgh, PA (US); John Bell, Ottawa (CA); Ragunath Singaravelu, Ottawa (CA); Carolina Ilkow, Ottawa (CA)

(21) Appl. No.: 18/565,727

(22) PCT Filed: Jun. 1, 2022
(86) PCT No.: PCT/CA2022/050878

§ 371 Date: Nov. 30, 2023

Related U.S. Application Data
(60) Provisional application No. 63/195,419, filed on Jun. 1, 2021.

(51) Int. Cl. A61K 48/00; A61K 38/45; A61K 45/06; A61P 35/00; C12N 15/86

(52) U.S. Cl. CPC A61K 48/005; A61K 38/45; A61K 45/06; A61P 35/00; C12N 15/86; C12Y 207/07; C12N 2710/24132; C12N 2710/24143

(57) ABSTRACT

A method of constitutively expressing a bacterial dinucleotide cyclase in a mammalian cell is provided. The method comprises transfecting the mammalian cell with a transgene encoding the bacterial dinucleotide cyclase and subjecting the mammalian cell to suitable growth conditions. In an embodiment, the bacterial dinucleotide cyclase is expressed in a tumour or cancer cell and is useful to treat cancer.

### FIELD OF THE INVENTION

The present invention generally relates to bacterial dinucleotide cyclases, and their therapeutic utility in mammals.

### BACKGROUND

The STING pathway is a major innate immune defence against bacteria, DNA viruses and tumour cells. In mammals, the STING signalling pathway plays a very important role in anti-microbial immunity and genome integrity. STING evolved to bind a family of secondary messenger molecules called cyclic dinucleotides (CDNs). These molecules are ubiquitous in the bacterial world where bacteria use them as important signalling molecules to regulate many important life processes. Bacteria have specialized enzymes, known as dinucleotide cyclases that generate CDNs. During bacterial infections, STING recognizes and binds to microbial CDNs, and thus initiates signal transduction cascades that drive production of type I interferons and other pro-inflammatory molecules needed to fend off bacterial infections. Cyclic dinucleotides are also ligands for the mammalian proteins ERAdP, which like STING, initiates an anti-microbial immune response when activated by these molecules.

Mammalian cells also have a dinucleotide cyclase termed cGAS. This enzyme catalyses the synthesis of cyclic GMP-AMP (cGAMP) upon detection of pathogen-derived or self DNA in the cytoplasm. Endogenous cGAMP similarly stimulates immune responses by binding and activating STING. Inappropriate appearance of nucleic acids in the cytosol of malignant cells with damaged DNA can also activate cGAS production of cGAMP. In fact, cyclic dinucleotides, such as cGAMP, are continuously exported by cancer cells and can mediate tumor immunogenicity. Tumor-derived cGAMP is transferred to tumor immune infiltrate and can drive type I IFN signaling in the immune compartment, including dendritic cells. Type I IFN signaling has been shown to play a critical role in driving anti-cancer immunity.

Recent work suggests driving cyclic dinucleotide uptake in endothelium also contributes to the success of STING agonist-based therapeutics.

As a result, many human cancers acquire deficiencies in the cGAS-STING signalling pathway in order to evade immune detection and are also more susceptible to infection by oncolytic DNA viruses than are normal cells.

STING agonized CDNs have the potential to increase the immunogenicity of tumors. In pre-clinical models, intratumoral injection of synthetic CDNs has been shown to drive a systemic anti-cancer response. Thus, synthetic CDNs have garnered significant interest as anti-cancer therapeutics, with a few STING agonist candidates reaching clinical development. However, limited efficacy and adverse effects have been observed in clinical trials which has been attributed, in part to, systemic STING activation in non-cancer tissue yielding poor patient tolerance. Furthermore, limited clinic indications remain applicable for STING agonist as they are primarily administered intratumorally.

It would be desirable to develop methods that utilize bacterial cyclic dinucleotides therapeutically.

### SUMMARY

The present invention identifies bacterial dinucleotide cyclases that can be expressed in mammalian cells and remain constitutively functional in the cytoplasm of these cells providing a continuous production of cyclic dinucleotides. In addition, transgenes expressing bacterial dinucleotide cyclases can be used therapeutically by introduction of these transgenes into target mammalian cells, for example, into tumour cells to boost immunogenicity of such cells.

Thus, in one aspect of the invention, a method of constitutively expressing a bacterial dinucleotide cyclase in a mammalian cell is provided comprising the step of introducing a transgene encoding the bacterial dinucleotide cyclase into the mammalian cell and subjecting the mammalian cell to suitable growth conditions.

In another aspect of the invention, a method of expressing a bacterial dinucleotide cyclase in a cancer or tumour cell is provided comprising administering a transgene encoding the dinucleotide cyclase to the cancer or tumour cell.

In another aspect of the invention, a method of treating cancer in a mammal is provided comprising administering a vector expressing the dinucleotide cyclase to the mammal.

In a further aspect of the invention, an oncolytic viral vector expressibly incorporating a transgene encoding a bacterial dinucleotide cyclase is provided.

These and other aspects of the invention are described in detail herein by reference to the following figures.

### BRIEF DESCRIPTION OF THE FIGURES

FIG. 1 graphically illustrates the activity of various bacterial cyclases in mammalian cells;

FIG. 2 graphically illustrates the activity of bacterial cyclase by the bacterial cyclase-expressing vaccinia viruses (A), and STING signaling in dendritic cells by the bacterial cyclase-expressing vaccinia viruses (B);

FIG. 3 illustrates that dinucleotide cyclase-expressing vaccinia strains activate STING in the absence of cGAS;

FIG. 4 graphically illustrates the expression of dinucleotide cyclases in A) RNA viruses and (B/C) DNA viruses to achieve activation of IFN signaling in reporter cells;

FIG. 5 is a schematic of a cyclic di-AMP ELISA;

FIG. 6 graphically illustrates cyclase activity in lysates (A) and supernatant (B) from cells transfected with plasmids expressing c-di-AMP, and cells infected with virus modified with plasmids expressing c-di-AMP;

FIG. 7 graphically illustrates reduction of tumour growth in vivo using cyclase-expressing oncolytic virus (A/B); and enhanced survival in a cancer model;

FIG. 8 graphically illustrates enhanced survival in a cancer model by treatment with a cyclase-expressing oncolytic virus;

FIG. 9 graphically illustrates survival in a cancer model by treatment with a cyclase-expressing oncolytic virus combined with a checkpoint inhibitor;

FIG. 10 graphically illustrates that virus expressing dinucleotide cyclase drives A) IFN signaling and B) RSAD2 expression in STING-active cells;

FIG. 11 further shows that viruses expressing dinucleotide cyclases drive IFN signaling in STING-active cells;

FIG. 12 graphically illustrates that infection of cancer cells with vaccinia expressing dinucleotide cyclase enables robust activation of IFN signaling;

FIG. 13 illustrates that virus expressing dinucleotide cyclase can drive IFN signaling in immune cells;

FIG. 14A)-N) illustrate nanostring results showing that both c-di-AMP and 2′3′-cGAMP are agonists of STING as evidenced by induction of a variety of inflammatory genes in RNA obtained from DC cultures treated with either c-di-AMP or 2′3′-cGAMP;

FIG. 15 provides SDS PAGE results showing that both c-di-AMP and 2′3′-cGAMP are agonists of STING in DC treated with either c-di-AMP or 2′3′-cGAMP; and

FIG. 16 graphically illustrates c-di-AMP and 2′3′-cGAMP are agonists of STING as evidenced by induction of nitrite and IFNß in DC cultures treated with either c-di-AMP or 2′3′-cGAMP.

### DETAILED DESCRIPTION

In a first aspect, a method of achieving constitutive expression of bacterial dinucleotide cyclases in mammalian cells is provided.

The term “bacterial dinucleotide cyclase”, also referred to herein as “dinucleotide cyclase” and/or “cyclase”, refers to a bacterial enzyme that catalyzes the synthesis of cyclic dinucleotides such as c-di-GMP, c-di-AMP, and cGAMP, as well as c-UAMP, c-di-UMP, c-UGM, c-CUMP, and c-AAGMP. Dinucleotide cyclases, thus, include, but are not limited to, i) di-adenylyl cyclases (DAC) proteins that synthesize c-di-AMP, such as DisA, CdaA, and CdaS; ii) proteins containing GGDEF domains (Pfam family: PF00990) that synthesize c-di-GMP; and iii) CD-NTase enzymes that have the catalytic domain known as SMODS (PF18144) that synthesize 3′-5′ cGAMP, such as DncV.

Bacterial dinucleotide cyclases in accordance with the present invention are those cyclases which remain constitutively functional in the cytoplasm of mammalian cells, i.e. dinucleotide cyclases from pathogenic bacteria that survive at 37° C. and which retain activity as depicted by an OD reading of at least about 0.5 at 630 nm, indicative of SEAP activity and correlates with the activation level of interferon signaling induced by cyclic dinucleotides. Thus, dinucleotide cyclases in accordance with the invention retain at least about 20% of their endogenous activity, preferably at least about 30%, 40%, 50% or more of their endogenous (wildtype) activity when expressed in mammalian cells. Examples of suitable dinucleotide cyclases include c-di-GMP cyclases from Vibrio cholera such as VCA0848, and c-di-AMP cyclases such as CdaA from Listeria monocytogenes and MtbDisA from Mycobacterreium tuberculosis.

In a method of expressing a bacterial dinucleotide cyclase in a mammalian cell, a transgene encoding the dinucleotide cyclase is prepared using well-established gene synthesis techniques.

As will be appreciated by one of skill in the art, the transgene encoding a selected bacterial dinucleotide cyclase may incorporate nucleic acid encoding the endogenous cyclase, or may incorporate nucleic acid encoding a modified dinucleotide cyclase which retains activity to catalyze the synthesis of a cyclic dinucleotide, i.e. a functional dinucleotide cyclase domain. In this regard, modifications may be made in regions of the cyclase gene that do not adversely affect cyclase activity, for example, modifications in regions that do not encode amino acid residues or motifs that are essential to activity, for example, required for substrate binding or for dimer or multimer formation, or within a catalytic domain. Thus, the cyclase gene may be truncated or modified while retaining the catalytic domain intact, or may only incorporate the catalytic domain of the selected dinucleotide cyclase, provided that the gene retains dinucleotide cyclase activity. Codons within the endogenous cyclase gene sequence may also be optimized for expression in mammalian cells. For example, negative regulatory domains may be deleted, the dimerization domain may be substituted with a heterologous dimerization domain (in cyclases which dimerize or multimerize) and other optimizations may be included in the cyclase gene sequence that enhance the cyclase gene sequence for expression in mammalian cells. Codon optimization may be conducted using available software for this purpose.

A mammalian cell is then transfected with the transgene encoding the bacterial dinucleotide cyclase, either as a linear molecule, a covalently-closed linear construct, or mini-circle. Alternatively, the transgene is incorporated into a plasmid, cosmid or viral vector using methods known in the art for introduction into a mammalian cell. As used herein, the term mammalian cell includes any cell from a mammal. Thus, introduction of a cyclase transgene into mammalian cells may be conducted ex vivo to generate a cell for therapeutic use, or may be introduced into cells in vivo.

Constitutive expression of the dinucleotide cyclase is achieved when the mammalian cell is subjected to suitable growth conditions, i.e. conditions that correlate with in vivo cytoplasmic conditions.

In an embodiment, a method of expressing a bacterial dinucleotide cyclase in mammalian cells such as tumour, cancer or immune cells is provided comprising introducing a transgene encoding the dinucleotide cyclase into the tumour, cancer or immune cells. The transgene may be introduced into such cells by various transfection techniques including using chemical methods such as cationic polymers or calcium phosphate transfection, lipid-based methods (lipofection or liposome-based tranfection) or physical methods such as microinjection or electroporation. The transgene may also be introduced into tumour, cancer or immune cells using a vector such as a plasmid or cosmid adapted to expressibly incorporate the cyclase transgene. Example of immune cells include dendritic cells, macrophages, neutrophils, eosinophils, basophils, mast cells, monocytes, natural killer cells, and lymphocytes.

Expression of a bacterial dinucleotide cyclase in tumour, cancer or immune cells advantageously enhances the immunogenicity of these cells, due to the generation of STING and ERAdP ligands, and thereby positively alters the tumour microenvironment. The expressed cyclases can be designed to be secreted by the expressing tumour or cancer cells and/or designed to bind and enter specific immune cells (e.g. dendritic cells or macrophages) to stimulate the innate and acquired immune system in the tumour microenvironment and draining lymph nodes.

Alternatively, a viral vector incorporating the transgene, including replicating or non-replicating viral vectors, may be used to infect a mammalian cell such as a tumour, cancer or immune cell. Suitable DNA viral vectors adapted to expressibly incorporate the cyclase transgene, e.g. under the control of a viral promoter and including any other elements required for expression of the cyclase, include, for example, poxviruses such as vaccinia virus and modified vaccinia virus, adenoviruses, adeno-associated viruses, herpes simplex virus and cytomegalovirus, including various serotypes thereof, both replication-competent and replication-deficient. RNA viral vectors may also be adapted to expressibly incorporate a transcript of the cyclase transgene including, but not limited to, RNA viruses such as vesicular stomatitis viruses, retroviruses such as MoMLV, lentiviruses, Sendai viruses, measles-derived vaccines, Newcastle disease virus, alphaviruses such as Semliki Forest virus, flaviviruses, or an RNA replicon based on an RNA virus (i.e. derived from alphavirus, flavivirus, etc). Preferably, the viral vector is replication-competent.

In a further aspect of the invention, an oncolytic viral vector expressibly incorporating a transgene encoding a bacterial dinucleotide cyclase is provided. Expression of a bacterial cyclase from an oncolytic virus enhances the efficacy of the oncolytic virus as a cancer therapeutic in vivo. For example, lysis of cyclase-expressing tumour cells will yield a potent immunological stimulus (adjuvant), i.e. the dinucleotide cyclase to generate cyclic dinucleotides which stimulate the STING signaling, and provision of tumour antigens to the immune system.

An oncolytic virus expressing a selected dinucleotide cyclase may be prepared by incorporating a transgene encoding the cyclase into the virus using standard recombinant technology. For example, the transgene may be incorporated into the genome of the virus, or alternatively, may be incorporated into the virus using a plasmid incorporating the transgene. The present method is not particularly restricted with respect to the oncolytic virus that may be utilized and may include any replicating oncolytic virus capable of destroying tumour, while being appropriate for administration to a mammal. Examples of oncolytic viruses that may be utilized in the present method include both DNA and RNA oncolytic viruses, including but not limited to, rhabdoviruses such as vesiculoviruses, e.g. vesicular stomatitis virus (VSV) and Maraba viruses, Ephemerovirus, Cytorhabdovirus, Nucleorhabdovirus and Lyssavirus viruses, as well as measles, vaccinia, herpes, myxoma, parvoviral, Newcastle disease, adenoviral and semliki forest viruses.

A bacterial dinucleotide cyclase-expressing transgene, cells comprising the transgene, or vector incorporating the transgene, such as an oncolytic viral vector expressing a bacterial dinucleotide cyclase, is useful in a method to treat cancer in a mammal. The term “cancer” is used herein to encompass any cancer, including but not limited to, melanoma, sarcoma, lymphoma, carcinoma such as brain, head and neck, bladder, breast, cervical, prostate, lung, liver, renal cell, skin, rectal, stomach and colon cancer, and leukaemia. The term “mammal” refers to humans as well as non-human mammals.

The method includes administration to the mammal of the dinucleotide cyclase-expressing transgene, cells comprising the transgene, or a vector incorporating the transgene. The transgene, cells or vector, such as an oncolytic vector, is administered to the mammal in any one of several ways including, but not limited to, intravenously, intramuscularly, intratumorally, or intranasally. As will be appreciated by one of skill in the art, the transgene, cells or vector (e.g. oncolytic vector) will be administered in a suitable carrier, such as saline or other suitable buffer.

The transgene, cells or vector (e.g. oncolytic vector) is administered to the mammal in an amount sufficient to generate a tumour-reducing response, e.g. a response which results in statistically significant reduction of tumour growth. The determination of statistical significance is well-established in the art. Statistical significance is attained when a p-value is less than the significance level. In this regard, the tumour-reducing response is a reduction in tumour size and/or growth of at least 5% or more, e.g. 10%, 20%, 30% or more, such as 50% or more. As one of skill in the art will appreciate, the amount of oncolytic cyclase-expressing vector required to generate a tumour-reducing response will vary with a number of factors, including, for example, the selected dinucleotide cyclase, the oncolytic vector used to deliver the cyclase, and the mammal to be treated, e.g. species, age, size, etc. In this regard, for example, intratumoral administration of about 10⁷ to 10⁸ PFU of oncolytic vector to a mouse is sufficient to generate a tumour-reducing response. A corresponding amount would be sufficient for administration to a human to generate a response. For intravenous administration, a similar or greater dosage may be utilized, for example, a dosage of about 10-100 times greater.

The cyclase-expressing transgene, cells or vector, such as an oncolytic vector, may be administered to the mammal in conjunction with at least one additional therapeutic agent. The therapeutic agent may also be useful to treat cancer such as another anti-tumor agent, or may be an agent that facilitates the anti-cancer activity of the oncolytic vector. The additional therapeutic agent may also be an immunotherapy drug such as an immune checkpoint inhibitor which prevents the action of checkpoint protein of tumour cells to interfere with the activity of host immune cells. Checkpoint inhibitors may be antibodies. Examples of checkpoint inhibitors include CTLA-4 inhibitors such as ipilimumab, PD-1 inhibitors such as nivolumab and pembrolizumab, and PD-LI inhibitors such as atezolisumab and avelumab.

Embodiments of the invention are described in the following specific examples which are not to be construed as limiting.

### Example 1—Expression and Function of Bacterial Cyclases in Mammalian Cells

Candidate bacterial cyclases were selected from pathogenic bacteria that can live at 37° C. Wild-type and various mutated c-di-GMP and c-di-AMP cyclase genes were cloned into pcDNA3.1(+) and HEK 293T cells were transfected for 24 h with plasmids expressing each candidate using established methods. Lysates were prepared with hypotonic buffer and heated at 95 C for 5 min prior to transfer onto THP1-Blue-ISG reporter cells.

C-di-GMP cyclases from Vibrio cholerae were analyzed, including: (1) VC2285 (WT) (Accession number: WP_001052610.1); (2) VC2285 (R174A/D177A, E393A); (3) VCA0848 (WT) (Accession number: WP_000998604.1); (4) VCA0848 (R273A/D276A); (5) VCA0848 (human albumin preproprotein signal peptide added at N-terminus); from Pseudomonas aeruginosa, including (6) PA2771(Dcsbis, WT) (Accession number: WP_003114426.1); (7) PA2771(Dcsbis, E10A, R97A/D100A, R137A/D140A, R207A/D210A, R225A/D228A, R233A/D236A, R251A/D254A); (8) PA3702 (WspR, WT) (Accession number: WP_003103734.1); (9) PA3702 (WspR, N-terminal 1-175 replaced with Saccharomyces cerevisiae GCN4(249-278)); (10) PA3702 (WspR, N-terminal 1-175 replaced with human albumin, preproprotein signal peptide and Saccharomyces cerevisiae GCN4(249-278)) and from Escherichia coli, (11) ECSP_2022 (ydeH, WT) (Accession number: WP_000592852.1) and (12) ECSP_2022 (ydeH,R56A/D59A, R62A/D65A,R73A/D76A, R141A/D144A).

C-di-AMP cyclases were also analyzed, including: (13) CdaA (WT) (Accession number: WP_003722380.1) and (14) CdaA (N-terminal 1-80 deletion) from Listeria monocytogenes, and (15) MtbDisA(WT) (Accession number: WP_003900111.1) from Mycobacterium tuberculosis. The following controls were used: (16) Mock; (17) GFP; (18) c-di-AMP; and (19) c-di-GMP.

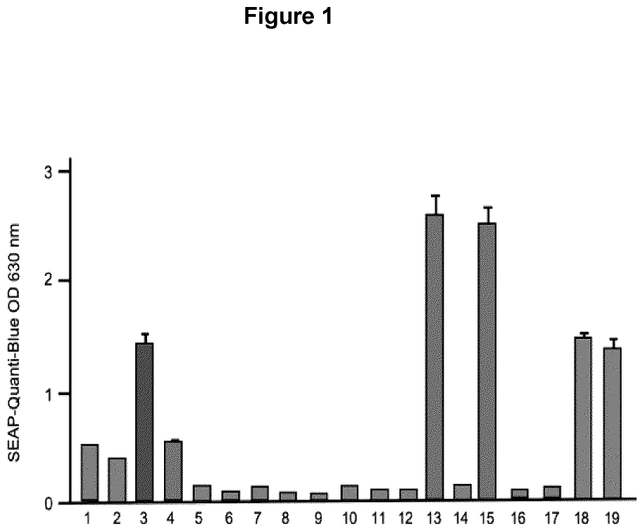

SEAP activity was quantified following 24 h incubation of THP1-Blue-ISG reporter cells with lysates from bacterial dinucleotide cyclase plasmid-transfected HEK 293T cells. Increased SEAP activity correlates with the activation levels of interferon signaling induced by cyclic dinucleotides present in lysates. VCA0848 (Vibrio cholera, c-di-GMP) (3), CdaA (Listeria monocytogenes, c-di-AMP) (13) and MtbDisA (Mycobacterium tuberculosis, c-di-AMP) (15) all displayed activity when expressed in mammalian cells as shown in FIG. 1.

### Example 2—STING Signaling in Dendritic Cells by the Bacterial Cyclase-Expressing Vaccinia Viruses

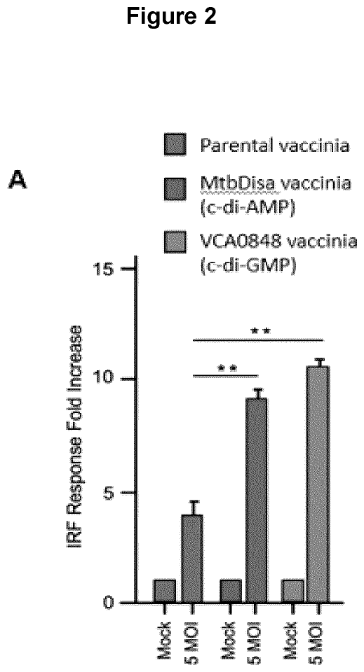

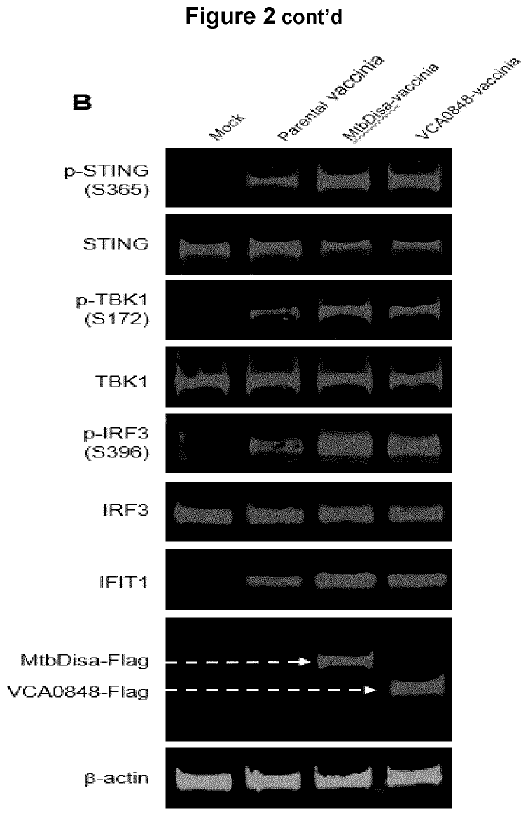

The bacterial dinucleotide cyclases, MtbDisA and VCA0848 were expressed from an oncolytic vaccinia virus. THP1-Blue-ISG cells were infected with the parental vaccinia virus or with the bacterial cyclase MtbDisA or VCA0848-expressing virus for 24 h. The supernatants were then rendered for Quanti-blue colorimetry using Quanti-Blue reagent according to the manufacturer's instructions. The production of SEAP was enhanced, as measured by IRF response, when either dinucleotide cyclase was expressed by the virus as shown in FIG. 2A.

Wild-type (WT) murine dendritic cells (DCs) derived from C57B16 mice were infected with the parental vaccinia virus or with the bacterial cyclase MtbDisA or VCA0848-expressing virus for 24 h. The whole cell lysates were prepared using RIPA buffer and rendered for Western blot analysis of the STING signaling axis, i.e., the phosphorylation of STING, TBK1 and IRF3, as well as the induction of its downstream target gene IFIT1.

Activation of STING and potentiated downstream signaling (i.e. activation of IRF3) and upregulation of interferon-responsive genes such as IFIT1 was substantially enhanced by the cyclase-expressing viruses as compared to the effect of the parental vaccinia virus as shown in FIG. 2B.

### Example 3—cGAS not Required for Activation of STING by Cyclase-Expressing Viruses

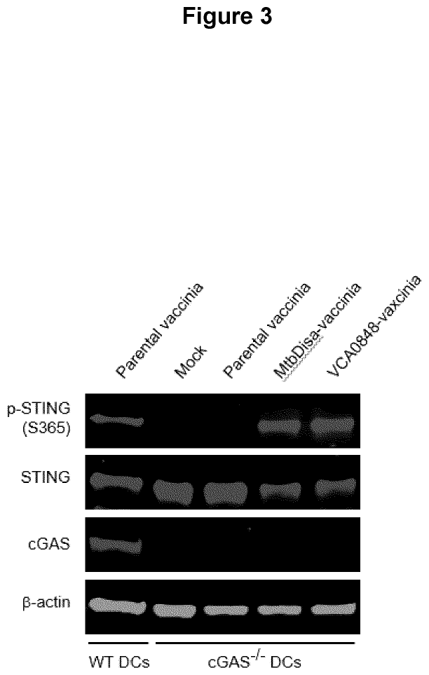

Murine dendritic cells (DCs) were infected for 24 h with the parental or MtbDisA- or VCA0848-expressing vaccinia strains. Whole cell lysates were prepared using RIPA buffer and rendered for Western blot analysis for STING phosphorylation. While the parental vaccinia could activate STING in wild-type DCs, it failed to do so in cGAS−/− DCs. In contrast, both dinucleotide cyclase-expressing vaccinia strains were able to activate STING in the absence of cGAS. Results are shown in FIG. 3.

### Example 4—Viral Expression of Dinucleotide Cyclases

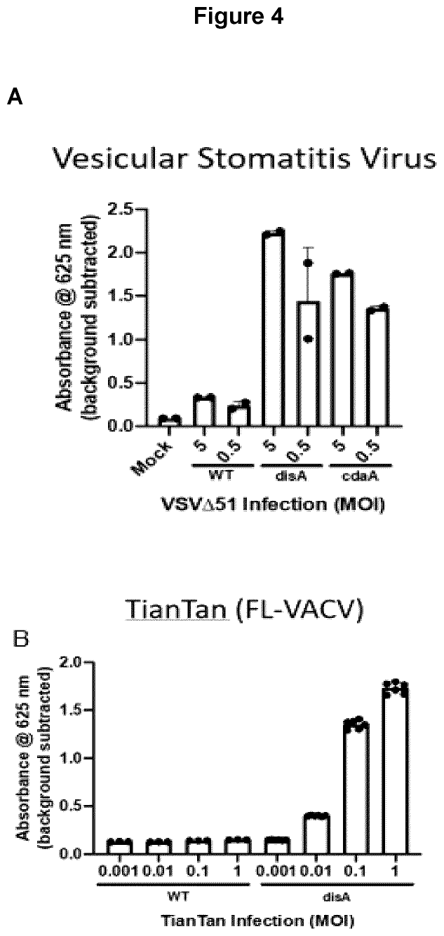

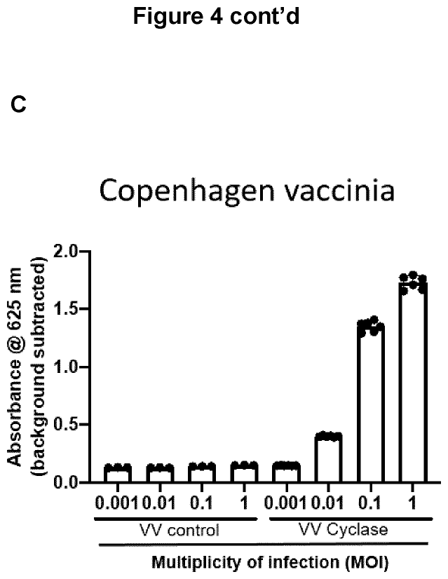

Hek293-Dual™ hSTING-H232 cells, expressing an interferon regulatory factor (IRF) driven SEAP reporter, were infected with VSV, or Vaccinia strains (Copenhagen or TianTan), wild-type or strains expressing disA or CdaA at various MOIs. SEAP assay was performed using Quanti-Blue colorimetric assay (Invivogen) as per manufacturer's protocols to analyze the amount of IFN signalling induction. The results demonstrate that dinucleotide cyclases were expressed from either RNA viruses, exemplified by VSV (FIG. 4A), or DNA viruses, such as strains of vaccinia, e.g. TianTan (FIG. 4B) and Copenhagen (FIG. 4C), leading to enhanced activation of IFN signaling in reporter cells.

### Example 5—Detection of c-Di-AMP in Transfected Virus and Viral-Infected Cells

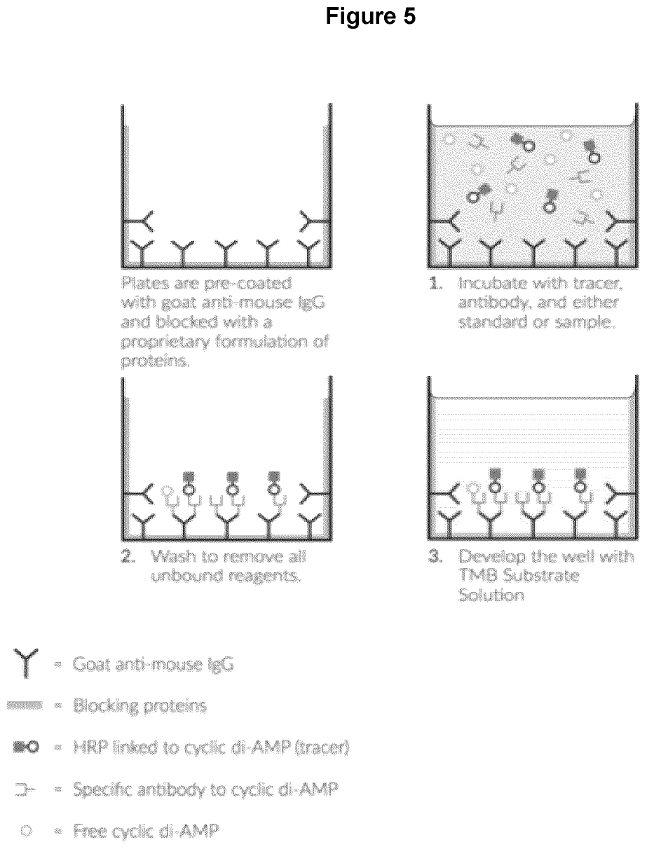

HeLa cells were infected with Vaccinia-Mtb-disA or parental virus (MOI=0.1) for 48 hours. As a positive control, cells were transfected with expression vector for Mtb-disA. Cells were lysed using mammalian protein extraction reagent (M-PER; Fisher-Scientific), as per manufacturer's protocol. Supernatants and cell lysates were boiled at 90 degrees for 15 minutes and then cooled on ice. ELISA for detection of cyclic di-AMP levels was performed as per manufacturer's protocol using commercial kit (Cayman Chemicals Cat #: 501960). An overview of the cyclic di-AMP ELISA is shown in FIG. 5.

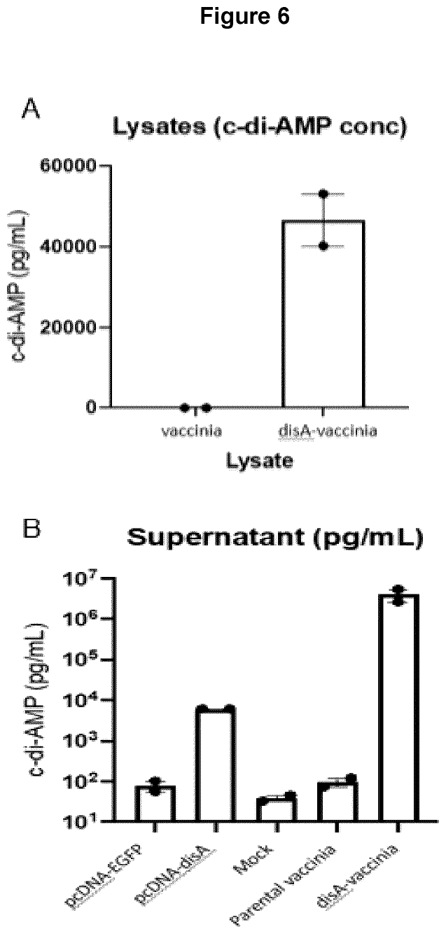

The results show that c-di-AMP was detected by ELISA in lysates and supernatants from cells transfected with plasmids expressing c-di-AMP, or cells infected with virus genetically modified with plasmids expressing c-di-AMP (see FIG. 6A/B). Thus, disA-expressing vaccinia virus actively produces cyclic di-AMP in infected cells. In the tumor microenvironment, released cyclic di-AMP enables stimulation of STING signaling in bystander immune cells and endothelium, to promote an anti-cancer immune response.

### Example 6—Enhanced Tumour Control by a Cyclase-Expressing Virus In Vivo

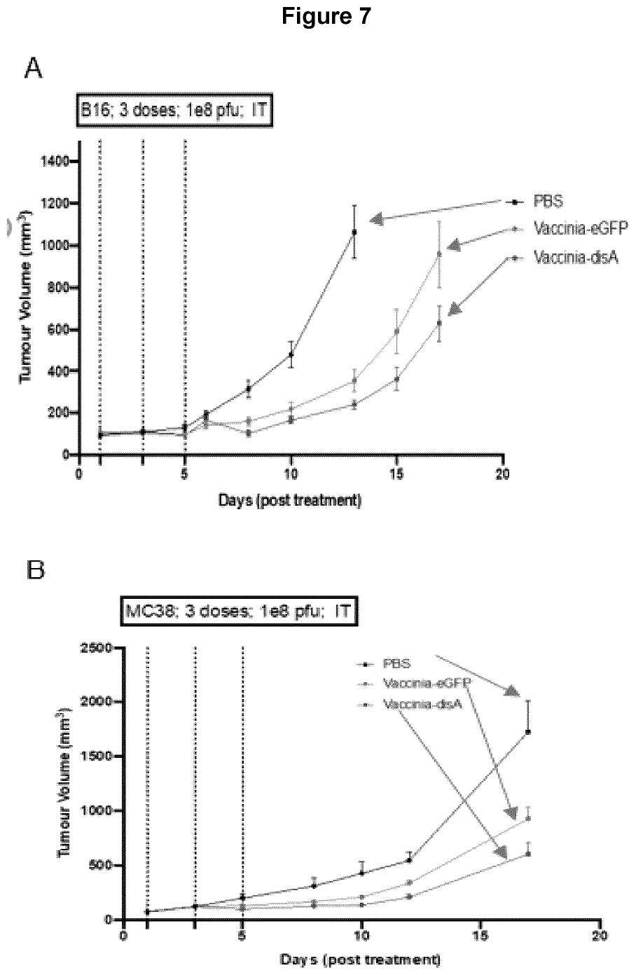

Mice were engrafted subcutaneously with either B16 melanoma or MC38 colon carcinoma cells. Mice with resulting tumours were then injected intratumourally with PBS, parental vaccinia or vaccinia-Mtb-DisA (1×108 pfu). Tumour volumes were measured at various timepoints with calipers and average volumes are displayed. Mice treated with DisA-expressing oncolytic virus displayed superior tumour control (reduced tumour growth) in both melanoma (FIG. 7A) and colon carcinoma (FIG. 7B) tumour models, relative to untreated and wild-type viral controls.

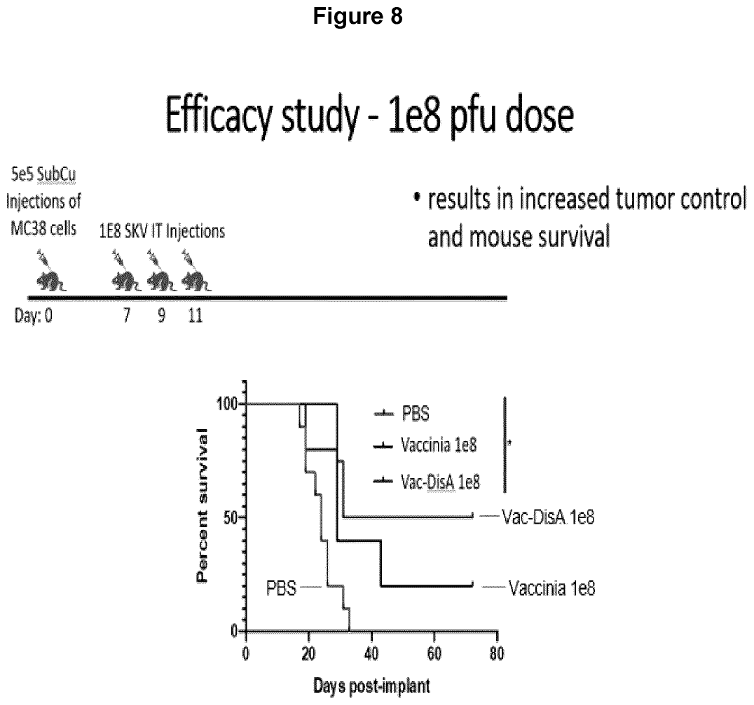

In addition, mice with colon carcinoma tumour receiving the MtbDisA-expressing oncolytic virus displayed enhanced survival (see FIG. 8).

The data demonstrates that oncolytic virus expression of dinucleotide cyclases drives a potent anti-tumor immune response.

### Example 7—Tumour Control with Cyclase-Expressing Virus Combined with Checkpoint Inhibitor

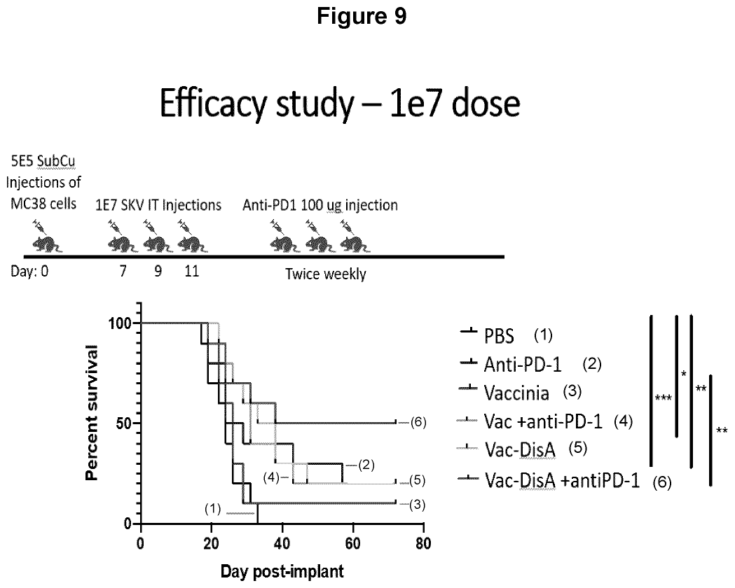

Mice were engrafted subcutaneously with 5×10⁵ MC38 colon carcinoma cells and subsequently treated with systemic checkpoint inhibitor, anti-PD-1 antibody alone (100 ug), oncolytic Vaccinia virus alone (1×10⁷ plaque forming units), checkpoint inhibitor plus Vaccinia virus, Vaccinia expressing MtbDisA and Vaccinia expressing MtbDisA plus anti-PD-1. As shown in FIG. 9, mice treated with Vaccinia expressing MtbDisA plus anti-PD-1 displayed enhanced survival. The data demonstrates that oncolytic virus expression of dinucleotide cyclases drives synergistic survival combined with a checkpoint blockade.

### Example 8—In Vitro Characterization of Vaccinia Virus Expressing Dinucleotide Cyclase

HT29 human colorectal cancer cell lines were infected with Vaccinia expressing cyclases (Mtb-disA or VCA0848) and qPCR was performed to measure RSAD2 and IFNB1 expression 48 hours-post infection. RSAD2 is an interferon stimulated gene, whereas IFNB1 is a gene encoding for a type I IFN. Both are generally expressed downstream of STING signaling activation.

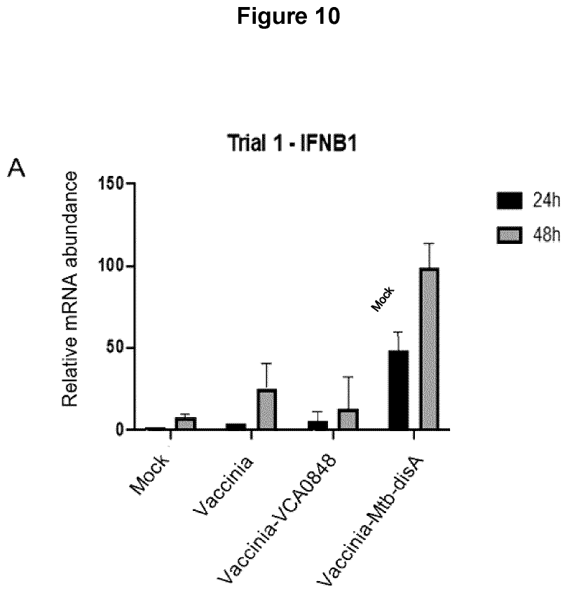

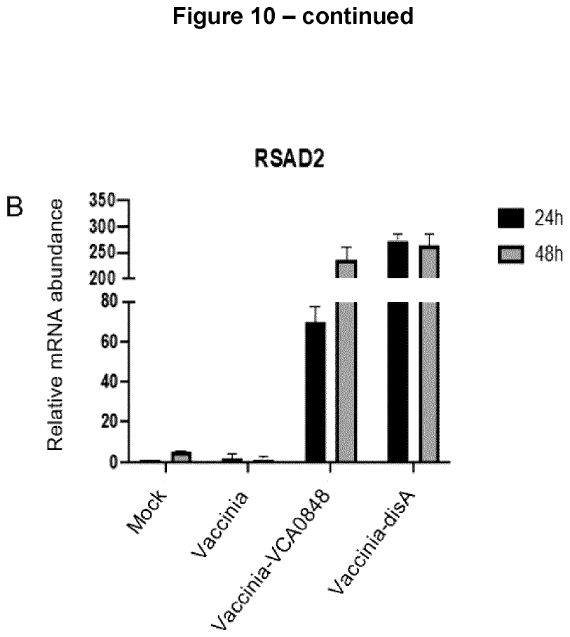

HT29 cells infected with Vaccinia expressing cyclases express both IFNB1 and RSAD2, and thus, possess active STING signaling (see FIG. 10). The data demonstrates that viruses expressing dinucleotide cyclases drive IFN signaling in STING-active cells.

### Example 9—In Vitro Characterization of Vaccinia Virus Expressing Dinucleotide Cyclase

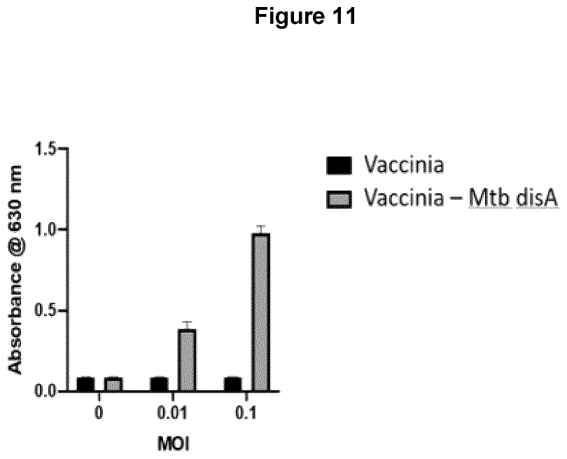

293-Dual™ hSTING-H232 cells expressing an interferon regulatory factor (IRF)-inducible SEAP reporter were infected with Vaccinia expressing Mtb-disA or parental virus at two MOIs (0.1 or 0.01). SEAP assay was performed using Quanti-Blue colorimetric assay (Invivogen) as per manufacturer's protocols. The data confirms that viruses expressing dinucleotide cyclases drive IFN signaling in STING-active cells (FIG. 11).

### Example 10—Interferon Expression in Tumour Infected with Virus Expressing Dinucleotide Cyclase

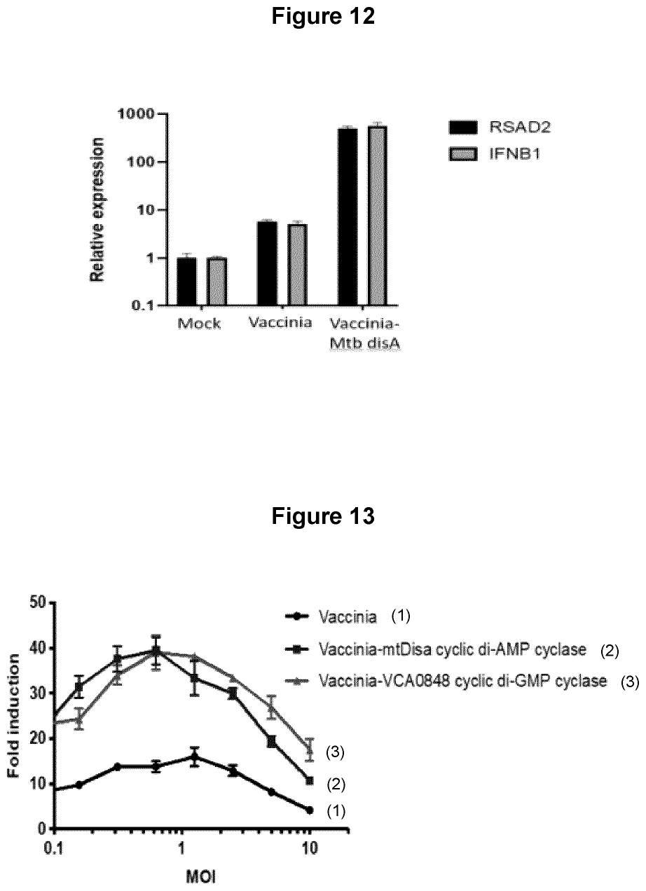

Low-grade serous carcinoma (LGSC) patient tumor cores were infected ex vivo with Vaccinia expressing dinucleotide cyclase (Mtb disA) or parental Vaccinia virus. 48 hours post-infection, RNA was harvested using Aurum™ Total RNA isolation kit (Bio-Rad) and qPCR analysis was performed to measure expression of RSAD2 and interferon production (IFNB1). The data shows that infection of cancer cells (likely STING-deficient) with vaccinia expressing dinucleotide cyclases enables robust activation of IFN signaling and production in the tumor microenvironment (see FIG. 12).

### Example 11—Interferon Expression in Tumour Infected with Virus Expressing Dinucleotide Cyclase

THP1-Dual cells (Invivogen) expressing an interferon regulatory factor (IRF)-inducible SEAP reporter were differentiated into macrophages using PMA. Forty eight hours post-differentiation, cells were infected with Vaccinia expressing nucleotide cyclase or parental virus at a range of MOIs. The SEAP assay was performed using Quanti-Blue colorimetric assay (Invivogen) as per manufacturer's protocols. The data demonstrates that viruses expressing dinucleotide cyclases can drive IFN signaling in immune cells (see FIG. 13).

### Example 12—Signaling Triggered by Bacterial c-Di-AMP Versus Noncanonical 2′3′-cGAMP

Wild-type (sting+/+) and knock-out (Sting−/−) bone marrow-derived mouse DCs were prepared by culture of bone marrow cells in media containing GM-CSF (40 ng/ml). On day 7, the DCs in 4 ml at 1×10⁶ cells/ml were treated with either c-di-AMP (InvivoGen) or cGAMP (InvivoGen) at 5 μg/ml for various lengths of time as indicated. Whole cell lysates were prepared using RIPA buffer and rendered for SDS-PAGE. Following transfer, the membranes were then probed for IFIT1, iNOS and STING using relevant antibodies.

Nitrite and IFNß assays were also conducted. Supernatants were collected at 0 time and 24 h post-treatment and were rendered for analysis by using Griess Assay (Promega) and mouse IFNß ELISA (R & D Systems) kits, respectively.

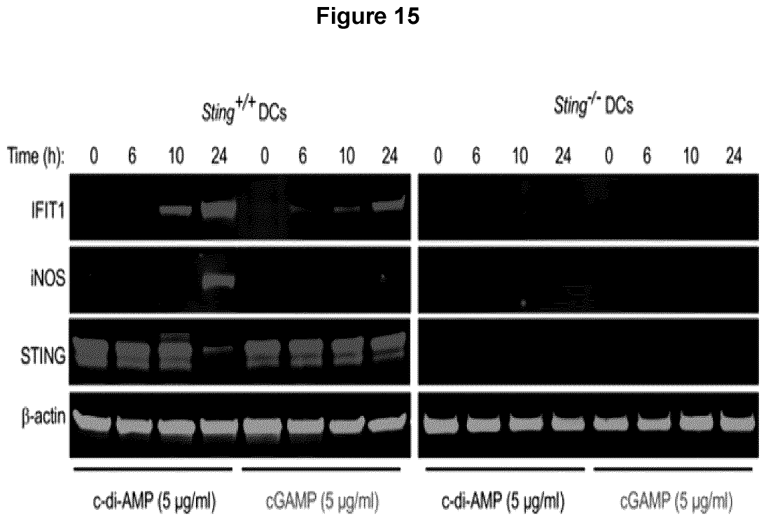

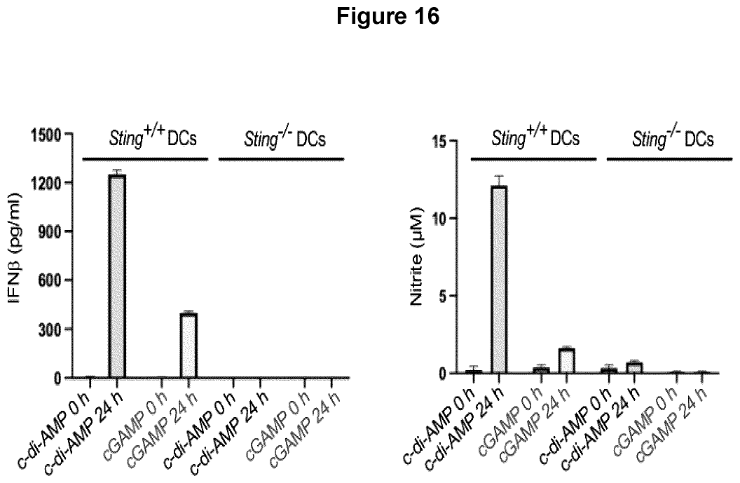

As shown in FIGS. 15 and 16, both c-di-AMP and 2′3′-cGAMP are agonists of STING with c-di-AMP exhibiting a stronger activation of the pathway as evidenced by more potent induction of nitrite and IFNß.

### Example 13—Nanostring Study

Wild-type (sting++) bone marrow-derived mouse DCs were prepared by culture of bone marrow cells in media containing GM-CSF (40 ng/ml). On day 7, the DCs in 4 ml at 1×10⁶ cells/ml were treated with either c-di-AMP (InvivoGen) or cGAMP (InvivoGen) at 5 μg/ml for various times as indicated. Total RNAs were isolated using RNAeasy Mini Kit (QIAGEN). The isolated RNA samples were then subjected to Nanostring analysis (Mouse PanCancer Immune Profiling as well as Mouse Immunology panels). Data was analyzed using nSolver software (normalized to housekeeping genes and untreated/0 hr samples.

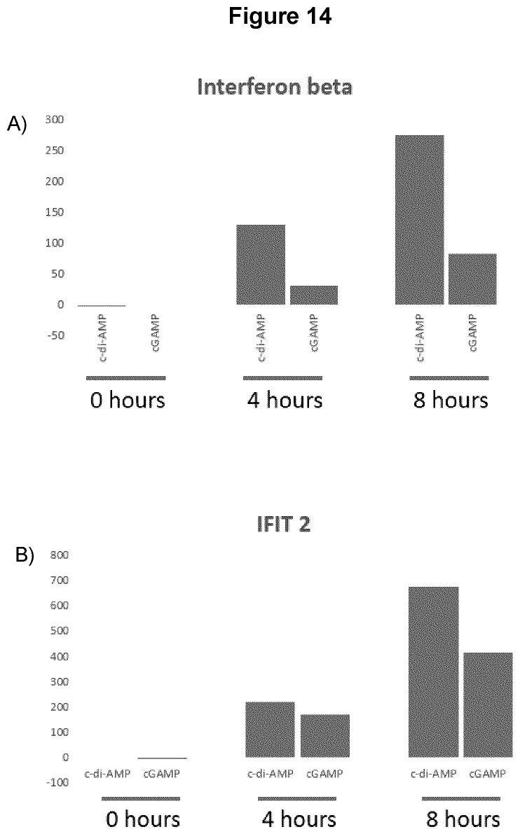

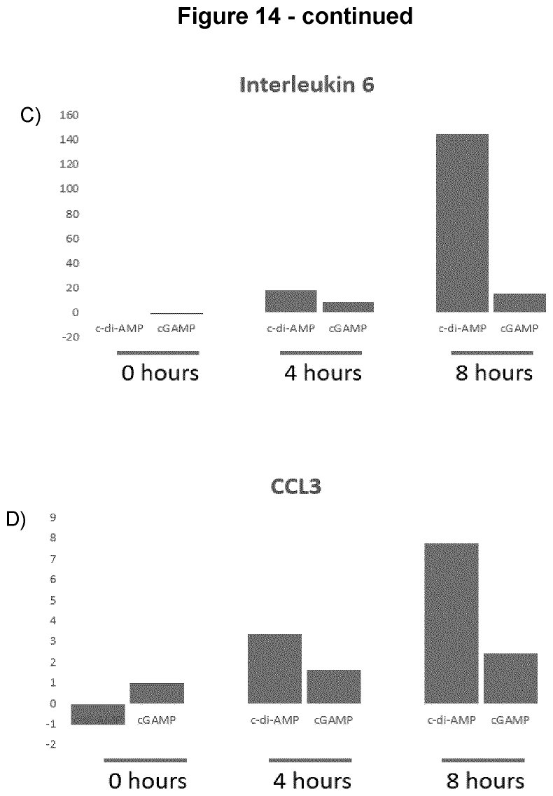

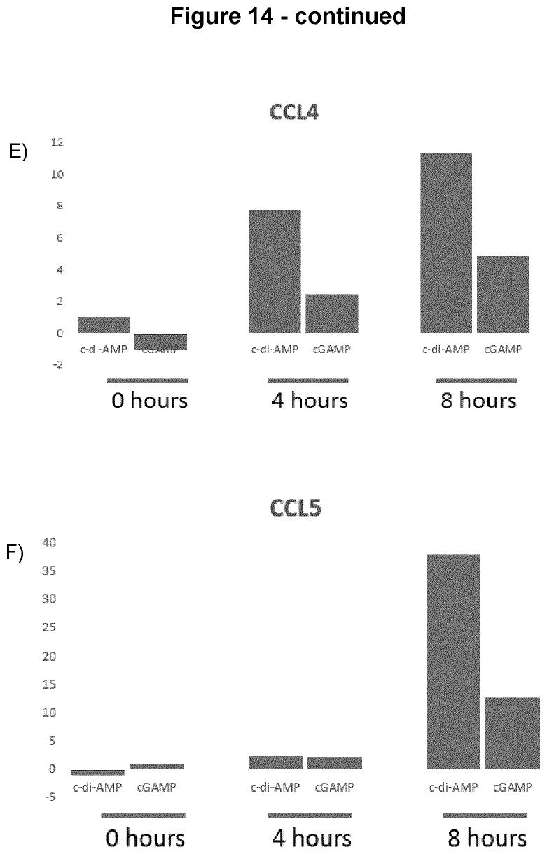

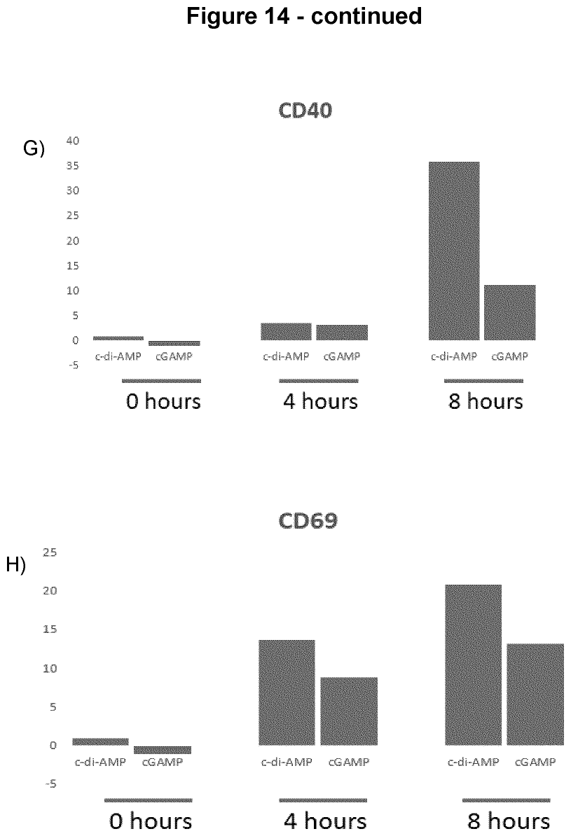

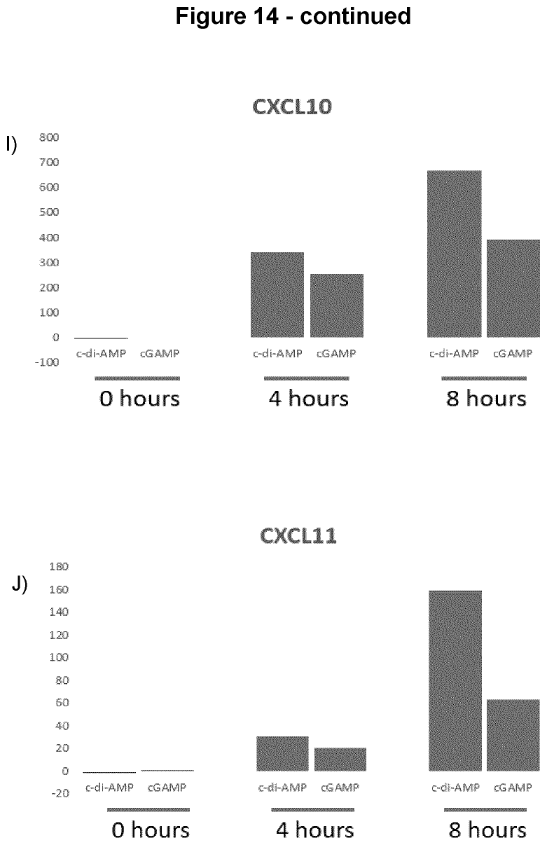

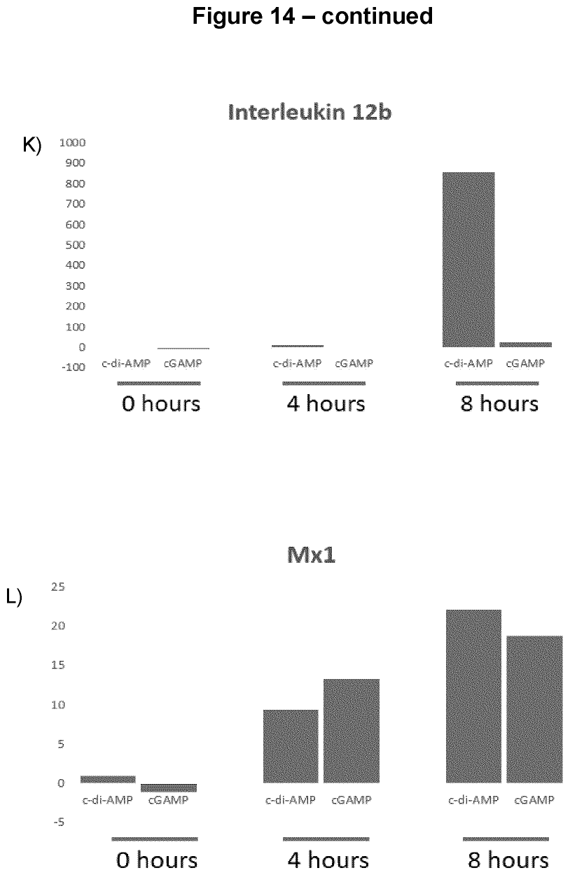

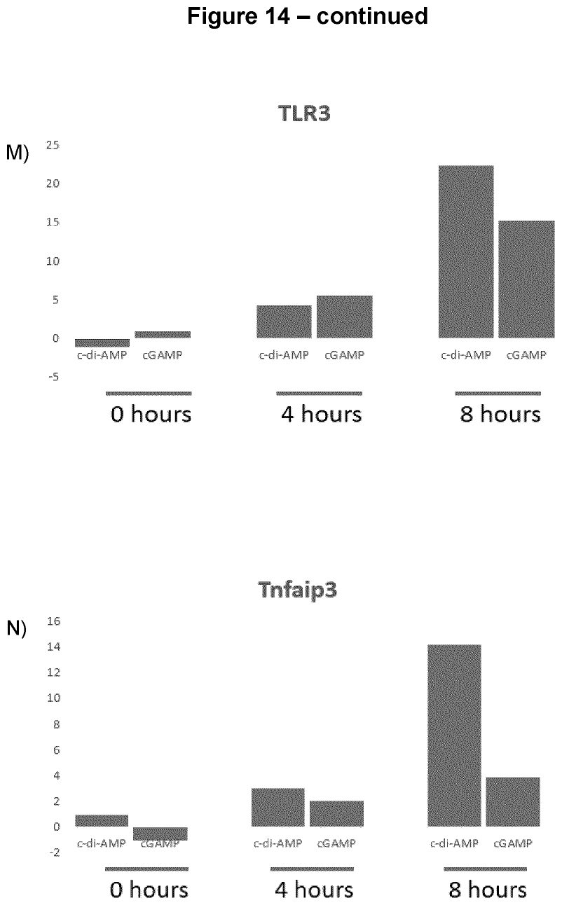

The results, in the form of raw transcript counts are provided in Table 1 below, and illustrated in FIG. 14.

TABLE 1

| | c-di- | | c-di- | | c-di- | |
|---|---|---|---|---|---|---|
| | AMP | cGAMP | AMP | cGAMP | AMP | cGAMP |
| | 0 hr | 0 hr | 4 hr | 4 hr | 8 hr | 8 hr |
| CCL3 | −1.01 | 1.01 | 3.36 | 1.65 | 7.75 | 2.44 |
| CCL4 | 1.02 | −1.02 | 7.77 | 2.46 | 11.33 | 4.92 |
| CCL5 | −1.01 | 1.01 | 2.46 | 2.24 | 38.02 | 12.89 |
| CD40 | 1.03 | −1.03 | 3.6 | 3.27 | 35.93 | 11.21 |
| CD69 | 1.04 | −1.04 | 13.73 | 8.85 | 20.83 | 13.24 |
| CXCL10 | −1.07 | 1.07 | 342.95 | 255.6 | 667.04 | 391.91 |
| CXCL11 | −1.43 | 1.43 | 31.09 | 21.28 | 159.73 | 63.19 |
| IFIT2 | 1.22 | −1.22 | 222.55 | 173.22 | 678.21 | 416.48 |
| IFNbeta | −1.37 | 1.37 | 130.51 | 31.01 | 274.07 | 82.69 |
| IL12b | 1.04 | −1.04 | 11.85 | 1.65 | 859.36 | 27.13 |
| IL6 | 1.06 | −1.06 | 18.17 | 9.09 | 145.15 | 15.75 |
| MX1 | 1 | −1 | 9.38 | 13.31 | 22.12 | 18.83 |
| NOS2 | 1.3 | −1.3 | 1.81 | 1.28 | 17.54 | 3.4 |
| TLR3 | −1.04 | 1.04 | 4.27 | 5.59 | 22.45 | 15.28 |
| Tnfaip3 | 1.01 | −1.01 | 3.09 | 2.05 | 14.25 | 3.93 |

The results show that both c-di-AMP and 2′3′-cGAMP are agonists of STING with c-di-AMP exhibiting a stronger activation as evidenced by more potent induction a variety of inflammatory genes in these DC cultures.

### Example 14—Dinucleotide Cyclase Sequences

The following sequences were used in the present work:

VCA0848:
DNA. Sequence (codon optimized):
(SEQ ID NO: 1)
ATGAACGATAAGGTCCTGGAGAGCGTGATTGAGATTACCGAACAG
AAAAATAGTCTGGCACTGAGCTACAGCATCCTGGCAACACTGTCT
GAGCTGCTGCCTCTGAGCACAGCCACACTGTTCCACCACCTGGGC
AGGAGCACCCTGATGGTGGCAAGACTGATCATCACAAAGAATGCA
GCAGGCAAGAAGGAGTATCAGTGGCAGTATGATCAGGTGTGCGCA
GATAATGGATACCAGCACTCCCAGTCTGAGATGGCCTTTTCCCAG
CAGGCCAATGGCCAGTATCAGTGCTTCTGTCCCATCCCTATCGAG
GAGCACTTTTCTGCCGAGCTGTGCCTGATCCTGAACAAGGACCCA
GAGCCCTACAGGATGCTGATCAACGGCTTCGCCAAGATCTACCGC
AATTATACCGTGATCCTGCACGAGTCTGAGAGAGACAAGCTGACA
GGCCTGCTGAATAGAAGAACACTGGAGGATCGGCTGAGACACACC
TTTGCCATCAACCCATCCACAGAGGAGAATCACAAGCTGTGGATC
GCCATGCTGGACATCGATCACTTCAAGGCCATCAATGACCACTTT
GGCCACATGATCGGCGATGAGATCCTGCTGATGTTCGCCCAGCAG
ATGCAGCACTATTTTGGACCTAGCTCCCAGCTGTTCCGGTTTGGC
GGCGAGGAGTTCGTGATCATCTTTTCTTCTGGAAATGAGCCACAG
ATCAAGCAGCAGCTGGATGGCTTCCGGCAGCAGATCAGGCGCCAC
AATTTTCCTAGGATTGGAGAGCTGAGCTTCTCCGCCGGATTTTGT
TCTCTGCGGCCCGGCGATTACCTGCCAACAATCCTGGACCACGCC
GATAAGGCCCTGTACTATGCAAAAGAGCACGGAAGGAACCAGGTG
CACTGCTATGAGCAGCTGTGCGAGAATGGCAAGATCGCCTCCGCC
CAGCGACCATTCTCCGATGATGTGGAACTGTTTGACTACAAGGAT
GACGATGATAAATAA
Amino acid sequence:
(SEQ ID NO: 2)
MNDKVLESVIEITEQKNSLALSYSILATLSELLPLSTATLFHHLG
RSTLMVARLIITKNAAGKKEYQWQYDQVCADNGYQHSQSEMAFSQ
QANGQYQCFCPIPIEEHFSAELCLILNKDPEPYRMLINGFAKIYR
NYTVILHESERDKLTGLLNRRTLEDRLRHTFAINPSTEENHKLWI
AMLDIDHFKAINDHFGHMIGDEILLMFAQQMQHYFGPSSQLFRFG
GEEFVIIFSSGNEPQIKQQLDGFRQQIRRHNFPRIGELSFSAGFC
SLRPGDYLPTILDHADKALYYAKEHGRNQVHCYEQLCENGKIASA
QRPFSDDVELFDYKDDDDK* (Flag-
tag on c-terminus)
MtbDisA from Mycobacterium tuberculosis:
DNA sequence (codon optimized:
(SEQ ID NO: 3)
ATGCACGCTGTGACAAGACCTACCCTGCGGGAGGCTGTGGCACGG
CTGGCACCTGGGACTGGACTGAGAGATGGACTGGAGAGAATCCTG
AGAGGAAGAACAGGCGCCCTGATCGTGCTGGGCCACGACGAGAAC
GTGGAGGCCATCTGCGACGGCGGCTTCAGCCTGGATGTGCGGTAC
GCCGCCACACGGCTGAGAGAGCTGTGCAAGATGGATGGAGCAGTG
GTGCTGTCCACAGATGGCAGCCGGATCGTGAGAGCAAATGTGCAG
CTGGTGCCAGACCCCTCTATTCCAACAGATGAGTCTGGAACCAGG
CACCGCTCCGCCGAGAGAGCCGCCATCCAGACAGGCTACCCTGTG
ATCAGCGTGTCCCACTCTATGAACATCGTGACCGTGTATGTGAGG
GGCGAGAGACACGTGCTGACCGACAGCGCCACCATCCTGTCTAGG
GCAAACCAGGCAATTGCCACACTGGAGAGGTATAAAACAAGACTG
GATGAGGTGTCCAGACAGCTGTCTAGAGCAGAGATCGAGGACTTT
GTGACACTGCGGGATGTGATGACAGTGGTGCAGAGACTGGAGCTG
GTGCGGAGAATTGGCCTGGTCATCGACTACGATGTGGTGGAGCTG
GGAACAGATGGCCGGCAGCTGAGACTGCAGCTGGACGAGCTGCTG
GGCGGCAACGATACAGCAAGGGAGCTGATCGTGCGCGATTATCAC
GCAAACCCAGAACCCCCTTCTACCGGGCAGATCAATGCCACCCTG
GATGAGCTGGATGCACTGTCTGATGGCGATCTGCTGGACTTCACA
GCCCTGGCAAAGGTGTTTGGATACCCAACCACAACCGAAGCACAG
GATAGCACCCTGTCCCCTAGGGGCTATCGCGCCATGGCCGGCATC
CCAAGGCTGCAGTTCGCCCACGCCGATCTGCTGGTGCGCGCCTTT
GGCACCCTGCAGGGCCTGCTGGCCGCCTCTGCCGGCGACCTGCAG
AGCGTGGATGGCATCGGCGCCATGTGGGCCCGGCACGTGAGAGAG
GGCCTGTCTCAGCTGGCAGAGTCCACCATTTCCGACCAGGATTAC
AAAGATGATGATGATAAGTAA
Amino acid sequence:
(SEQ ID NO: 4)
MHAVTRPTLREAVARLAPGTGLRDGLERILRGRTGALIVLGHDEN
VEAICDGGFSLDVRYAATRLRELCKMDGAVVLSTDGSRIVRANVQ
LVPDPSIPTDESGTRHRSAERAAIQTGYPVISVSHSMNIVTVYVR
GERHVLTDSATILSRANQAIATLERYKTRLDEVSRQLSRAEIEDF
VTLRDVMTVVQRLELVRRIGLVIDYDVVELGTDGRQLRLOLDELL
GGNDTARELIVRDYHANPEPPSTGQINATLDELDALSDGDLLDFT
ALAKVFGYPTTTEAQDSTLSPRGYRAMAGIPRLQFAHADLLVRAF
GTLQGLLAASAGDLQSVDGIGAMWARHVREGLSQLAESTISDQDY
KDDDDK* (FLAG at c-terminus)
Truncated CdaA from Listeria Monocytogenes
(put in TianTan and VSV)
(DFSNMSILHYLANIVDILVVWFVIYKVIMLIRGTKAVQLL
deleted from the N-terminus of CdaA):
DNA sequence:
(SEQ ID NO: 5)
ATGAAGGGCATCTTTATCATCATCGCTGTGAAGCTGCTGTCCGGA
TTCTTTGGCCTGCAGACCGTGGAGTGGATCACAGACCAGATGCTG
ACCTGGGGCTTCCTGGCCATCATCATCATCTTTCAGCCAGAGCTG
AGGCGCGCTCTGGAGACACTGGGAAGGGGCAACATCTTCACCCGC
TACGGAAGCAGGATCGAGAGAGAGCAGCACCACCTGATCGAGTCT
ATCGAGAAGAGCACCCAGTACATGGCCAAGAGGAGAATCGGCGCC
CTGATCTCCGTGGCTCGGGATACAGGAATGGACGATTACATCGAG
ACAGGCATCCCCCTGAACGCTAAGATCAGCTCCCAGCTGCTGATC
AACATCTTCATCCCCAACACACCTCTGCACGACGGAGCCGTGATC
ATCAAGGGCAACGAGATCGCTAGCGCCGCTTCCTACCTGCCACTG
AGCGATTCCCCCTTTCTGAGCAAGGAGCTGGGAACCAGGCACAGG
GCCGCTCTGGGCATCTCTGAGGTGACCGACAGCATCACAATCGTG
GTGTCTGAGGAGACAGGCGGAATCAGCCTGACCAAGGGCGGAGAG
CTGTTCCGCGACGTGAGCGAGGAGGAGCTGCACAAGATCCTGCTG
AAGGAGCTGGTGACCGTGACAGCCAAGAAGCCCTCTATCTTTAGC
AAGTGGAAGGGCGGAAAGTCCGAG
Amino acid sequence:
(SEQ ID NO: 6)
MKGIFIIIAVKLLSGFFGLOTVEWITDQMLTWGFLAIIIIFQPEL
RRALETLGRGNIFTRYGSRIEREQHHLIESIEKSTQYMAKRRIGA
LISVARDTGMDDYIETGIPLNAKISSQLLINIFIPNTPLHDGAVI
IKGNEIASAASYLPLSDSPFLSKELGTRHRAALGISEVTDSITIV
VSEETGGISLTKGGELFRDVSEEELHKILLKELVTVTAKKPSIFS
KWKGGKSE

Additional dinucleotide cyclase sequence information:

i) VC2285:
VCA2285 DNA sequence (codon optimized)-Flag:
(SEQ ID NO: 7)
ATGAATCTGAATAACTTCTCCCTGCGGTGGCTGACTACTCTGAAT
GCTCTGGCTGTGGTCCTGGGCTTTCTGATGTTTTACCTGACTTTC
AAGTATTTTTGGTCCCACGATAGAGAGGTGGCACAGGTGCTGCAG
CTGCAGCAGGCCGAGCTGCAGCGGGTGGAGACACTGCTGTCCCTG
GAGAGAAAGGCCATGGGAGCCTCTCTGGCAGACTATGCAGCATGG
GATGAGATGGCAGATTTCATCGCAAAGCCCACCCTGGAGTTTACA
CAGTCCAATATTGGAGAACACGCCTTCAGCTCCCAGTTTCTGGAC
GGCGTGTTCATCTACGATCCTGAGGGCAATCTGGTGTGGGGCAAG
AAGTATGATGCAGCAACAGGACAGTCTAGCTCCTATGAGCACCTG
CTGCCAGACTTTAGCAGAATCCTGCAGCAGGCAACCAGACTGTCT
GTGGATGAGATCAGCACATCCGTGCGGTACATGGTGGTGGAGGAT
GAACCATATCTGGCAGCAACAGCAAGAGTGTGCGACTCTGATGGC
AAGGGATGTAACAAGGGCTTCCTGATCTTTATCAAGAAGGTGAGG
GCCCAGTTTGCAAATGTGGTGGAGCAGGCAACAGGAGTGGATATT
GAGGTGCTGACATGCAAGAATGATGCCCCACTGCCACAGGATGAG
GTGGACGTGAGCTACATCAAGCAGCTGGACTATAGCGGCAACTCT
AGCGTGCTGTTCAAGATCAATCACCACATCAAGCACCCTCCCTTC
ATCAGAACAGAGGAGATCCTGGCCCTGCTGTTCTTTTCCCTGGTC
ATGTACCTGGTGAATCTGTGGGTGGTCATCGCCCTGATCAAGCCT
ATCACCACAGCCTCTCAGGTGCTGCAGCAGTTCAAGACCTCCGGC
GGCAAGATGCCAGATGCATCTACATTCATCTCCTCTGAGATGAAG
GAGTTTGCCACCACAATCAACAGAATTGTGGGACAGCTGGAGGAT
TCTCAGCAGGTGCTGAGGTGGCAGTCTGAGCACGACCCACTGACA
AGAATCTCCAATCGGAGACACCTGGAGAAGCAGCTGAAGTCTTAC
CTGTCTGACAGACCTCAGGCCTATCTGGTGCTGTTTCTGGTGGAC
ATCGATTTCTTTAAGCGGTTCAACGACAGCTTTGGCCACCTGGCA
GGCGATGAGGCCCTGTGCAGCGTGGCCGACGTGCTGCAGTCCGTG
GAGTTCCACGGAGAGAAGATTGTGGCAAGATTTGGCGGCGAGGAG
TTTTGCGTGGTGCTGGCATCTGACTGTGCCTTTGATGCCGAGCAG
TATGCCCAGCAGATGAGAAGCAAGATTGAGCAGCTGGCAATTGCA
AATCCTGTGGATGCACTGTGCCAGTACCTGACCGTGTCTATTGGC
GGCGTGTATGCAATCAGCCCTAAGATGGAGTCCTACCTGTCTCTG
TTCCACCAGGCAGACATGGCCCTGTACCACGCCAAGGAGCATGGG
AGAGACAGATACGTGGTGCGGAACTTCGTGGATTACAAAGATGAT
GATGATAAA
VCA2285 protein sequence-Flag:
(SEQ ID NO: 8)
MNLNNFSLRWLTTLNALAVVLGFLMFYLTFKYFWSHDREVAQVLQ
LQQAELQRVETLLSLERKAMGASLADYAAWDEMADFIAKPTLEFT
QSNIGEHAFSSQFLDGVFIYDPEGNLVWGKKYDAATGQSSSYEHL
LPDFSRILQQATRLSVDEISTSVRYMVVEDEPYLAATARVCDSDG
KGCNKGFLIFIKKVRAQFANVVEQATGVDIEVLTCKNDAPLPQDE
VDVSYIKQLDYSGNSSVLFKINHHIKHPPFIRTEEILALLFFSLV
MYLVNLWVVIALIKPITTASQVLQQFKTSGGKMPDASTFISSEMK
EFATTINRIVGQLEDSQQVLRWQSEHDPLTRISNRRHLEKQLKSY
LSDRPQAYLVLFLVDIDFFKRFNDSFGHLAGDEALCSVADVLQSV
EFHGEKIVARFGGEEFCVVLASDCAFDAEQYAQQMRSKIEQLAIA
NPVDALCQYLTVSIGGVYAISPKMESYLSLFHQADMALYHAKEHG
RDRYVVRNFVDYKDDDDK
ii) VCA2285 (R174A/D177A, E393A):
VCA2285 (R174A/D177A, E393A) DNA
sequence (codon optimized)-Flag:
(SEQ ID NO: 9)
ATGAATCTGAATAACTTCTCTCTGCGGTGGCTGACAACTCTGAAT
GCTCTGGCTGTGGTCCTGGGCTTTCTGATGTTCTACCTGACATTC
AAGTATTTTTGGTCCCACGATAGAGAGGTGGCACAGGTGCTGCAG
CTGCAGCAGGCCGAGCTGCAGCGGGTGGAGACACTGCTGTCCCTG
GAGAGAAAGGCCATGGGAGCCTCTCTGGCAGACTATGCAGCATGG
GATGAGATGGCAGATTTCATCGCAAAGCCCACCCTGGAGTTTACA
CAGTCCAATATTGGAGAACACGCCTTCAGCTCCCAGTTTCTGGAC
GGCGTGTTCATCTACGATCCTGAGGGCAATCTGGTGTGGGGCAAG
AAGTATGATGCAGCAACAGGACAGTCTAGCTCCTATGAGCACCTG
CTGCCAGACTTTAGCAGAATCCTGCAGCAGGCAACCAGACTGTCT
GTGGATGAGATCAGCACATCCGTGCGGTACATGGTGGTGGAGGAT
GAGCCTTATCTGGCAGCAACAGCAGCCGTGTGCGCATCTGATGGC
AAGGGATGTAACAAGGGCTTCCTGATCTTTATCAAGAAGGTGAGA
GCCCAGTTTGCAAATGTGGTGGAGCAGGCAACAGGAGTGGATATT
GAGGTGCTGACATGCAAGAATGATGCCCCACTGCCACAGGATGAG
GTGGACGTGAGCTACATCAAGCAGCTGGACTATAGCGGCAACTCT
AGCGTGCTGTTCAAGATCAATCACCACATCAAGCACCCTCCCTTC
ATCAGGACAGAGGAGATCCTGGCCCTGCTGTTCTTTTCCCTGGTC
ATGTACCTGGTGAATCTGTGGGTGGTCATCGCCCTGATCAAGCCT
ATCACCACAGCCTCTCAGGTGCTGCAGCAGTTCAAGACCTCCGGC
GGCAAGATGCCAGATGCATCTACATTCATCTCCTCTGAGATGAAG
GAGTTTGCAACAACAATCAACAGAATTGTGGGACAGCTGGAGGAT
TCTCAGCAGGTGCTGAGGTGGCAGTCTGAGCACGACCCACTGACA
AGAATCTCCAATCGGAGACACCTGGAGAAGCAGCTGAAGTCTTAC
CTGTCTGACAGACCACAGGCCTATCTGGTGCTGTTTCTGGTGGAC
ATCGATTTCTTTAAGAGATTCAACGACAGCTTTGGCCACCTGGCA
GGCGATGCCGCCCTGTGCAGCGTGGCCGACGTGCTGCAGTCTGTG
GAGTTCCACGGAGAGAAGATTGTGGCAAGGTTTGGCGGCGAGGAG
TTTTGCGTGGTGCTGGCATCTGACTGTGCCTTTGATGCAGAGCAG
TATGCACAGCAGATGCGCAGCAAGATTGAGCAGCTGGCAATTGCA
AATCCTGTGGATGCACTGTGCCAGTACCTGACCGTGTCTATTGGC
GGCGTGTATGCAATCAGCCCTAAGATGGAGTCCTACCTGTCTCTG
TTCCACCAGGCAGACATGGCCCTGTACCACGCCAAGGAGCATGGG
GGGACAGATACGTGGTGCGGAACTTCGTGGATTACAAAGATGATG
ATGATAAA
VCA2285
(R174A/D177A, E393A) protein sequence-Flag:
(SEQ ID NO: 10)
MNLNNFSLRWLTTLNALAVVLGFLMFYLTFKYFWSHDREVAQVLQ
LQQAELQRVETLLSLERKAMGASLADYAAWDEMADFIAKPTLEFT
QSNIGEHAFSSQFLDGVFIYDPEGNLVWGKKYDAATGQSSSYEHL
LPDFSRILQQATRLSVDEISTSVRYMVVEDEPYLAATAAVCASDG
KGCNKGFLIFIKKVRAQFANVVEQATGVDIEVLTCKNDAPLPQDE
VDVSYIKQLDYSGNSSVLFKINHHIKHPPFIRTEEILALLFFSLV
MYLVNLWVVIALIKPITTASQVLQQFKTSGGKMPDASTFISSEMK
EFATTINRIVGQLEDSQQVLRWQSEHDPLTRISNRRHLEKQLKSY
LSDRPQAYLVLFLVDIDFFKRFNDSFGHLAGDAALCSVADVLQSV
EFHGEKIVARFGGEEFCVVLASDCAFDAEQYAQQMRSKIEQLAIA
NPVDALCQYLTVSIGGVYAISPKMESYLSLFHQADMALYHAKEHG
RDRYVVRNFVDYKDDDDK
iii) VCA0848:
VCA0848 DNA sequence (codon optimized)-Flag:
(SEQ ID NO: 11)
ATGAACGATAAGGTCCTGGAGAGCGTGATTGAGATTACCGAACAG
AAAAATAGTCTGGCACTGAGCTACAGCATCCTGGCAACACTGTCT
GAGCTGCTGCCTCTGAGCACAGCCACACTGTTCCACCACCTGGGC
AGGAGCACCCTGATGGTGGCAAGACTGATCATCACAAAGAATGCA
GCAGGCAAGAAGGAGTATCAGTGGCAGTATGATCAGGTGTGCGCA
GATAATGGATACCAGCACTCCCAGTCTGAGATGGCCTTTTCCCAG
CAGGCCAATGGCCAGTATCAGTGCTTCTGTCCCATCCCTATCGAG
GAGCACTTTTCTGCCGAGCTGTGCCTGATCCTGAACAAGGACCCA
GAGCCCTACAGGATGCTGATCAACGGCTTCGCCAAGATCTACCGC
AATTATACCGTGATCCTGCACGAGTCTGAGAGAGACAAGCTGACA
GGCCTGCTGAATAGAAGAACACTGGAGGATCGGCTGAGACACACC
TTTGCCATCAACCCATCCACAGAGGAGAATCACAAGCTGTGGATC
GCCATGCTGGACATCGATCACTTCAAGGCCATCAATGACCACTTT
GGCCACATGATCGGCGATGAGATCCTGCTGATGTTCGCCCAGCAG
ATGCAGCACTATTTTGGACCTAGCTCCCAGCTGTTCCGGTTTGGC
GGCGAGGAGTTCGTGATCATCTTTTCTTCTGGAAATGAGCCACAG
ATCAAGCAGCAGCTGGATGGCTTCCGGCAGCAGATCAGGCGCCAC
AATTTTCCTAGGATTGGAGAGCTGAGCTTCTCCGCCGGATTTTGT
TCTCTGCGGCCCGGCGATTACCTGCCAACAATCCTGGACCACGCC
GATAAGGCCCTGTACTATGCAAAAGAGCACGGAAGGAACCAGGTG
CACTGCTATGAGCAGCTGTGCGAGAATGGCAAGATCGCCTCCGCC
CAGCGACCATTCTCCGATGATGTGGAACTGTTTGACTACAAGGAT
GACGATGATAAA
VCA0848 protein sequence-Flag:
(SEQ ID NO: 12)
MNDKVLESVIEITEQKNSLALSYSILATLSELLPLSTATLFHHLG
RSTLMVARLIITKNAAGKKEYQWQYDQVCADNGYQHSQSEMAFSQ
QANGQYQCFCPIPIEEHFSAELCLILNKDPEPYRMLINGFAKIYR
NYTVILHESERDKLTGLLNRRTLEDRLRHTFAINPSTEENHKLWI
AMLDIDHFKAINDHFGHMIGDEILLMFAQQMQHYFGPSSQLFRFG
GEEFVIIFSSGNEPQIKQQLDGFRQQIRRHNFPRIGELSFSAGFC
SLRPGDYLPTILDHADKALYYAKEHGRNQVHCYEQLCENGKIASA
QRPFSDDVELFDYKDDDDK
iv) VCA0848 (R273A/D276A): VCA0848 (R273A/D276A)
DNA sequence (codon optimized)-Flag:
(SEQ ID NO: 13)
ATGAACGACAAGGTCCTGGAGAGCGTGATTGAGATTACCGAGCAG
AAAAACAGCCTGGCCCTGAGCTACAGCATCCTGGCAACCCTGTCT
GAGCTGCTGCCTCTGAGCACAGCCACACTGTTCCACCACCTGGGC
AGGAGCACCCTGATGGTGGCAAGACTGATCATCACAAAGAATGCA
GCAGGCAAGAAGGAGTATCAGTGGCAGTATGATCAGGTGTGCGCA
GATAATGGATACCAGCACTCCCAGTCTGAGATGGCCTTTTCCCAG
CAGGCCAATGGCCAGTATCAGTGCTTCTGTCCCATCCCTATCGAG
GAGCACTTTTCTGCCGAGCTGTGCCTGATCCTGAACAAGGACCCA
GAGCCCTACAGGATGCTGATCAACGGCTTCGCCAAGATCTACCGC
AATTATACCGTGATCCTGCACGAGTCTGAGAGAGACAAGCTGACA
GGCCTGCTGAATAGAAGAACACTGGAGGATCGGCTGAGACACACC
TTTGCCATCAACCCATCCACAGAGGAGAATCACAAGCTGTGGATC
GCCATGCTGGACATCGATCACTTCAAGGCCATCAATGACCACTTT
GGCCACATGATCGGCGATGAGATCCTGCTGATGTTCGCCCAGCAG
ATGCAGCACTATTTTGGACCTTCTAGCCAGCTGTTCAGATTTGGC
GGCGAGGAGTTCGTGATCATCTTTTCTTCTGGAAATGAGCCACAG
ATCAAGCAGCAGCTGGATGGCTTCCGGCAGCAGATCAGGCGCCAC
AATTTTCCTAGGATTGGAGAGCTGAGCTTCTCCGCCGGATTTTGC
TCTCTGGCACCTGGGGCATACCTGCCAACAATCCTGGACCACGCC
GATAAGGCCCTGTACTATGCAAAAGAGCACGGAAGAAACCAGGTG
CACTGCTATGAGCAGCTGTGCGAGAATGGCAAGATCGCCTCCGCC
CAGAGACCATTTTCAGATGATGTGGAACTGTTTGATTACAAGGAC
GACGACGATAAA
VCA0848(R273A/D276A) protein sequence-Flag:
(SEQ ID NO: 14)
MNDKVLESVIEITEQKNSLALSYSILATLSELLPLSTATLFHHLG
RSTLMVARLIITKNAAGKKEYQWQYDQVCADNGYQHSQSEMAFSQ
QANGQYQCFCPIPIEEHFSAELCLILNKDPEPYRMLINGFAKIYR
NYTVILHESERDKLTGLLNRRTLEDRLRHTFAINPSTEENHKLWI
AMLDIDHFKAINDHFGHMIGDEILLMFAQQMQHYFGPSSQLFRFG
GEEFVIIFSSGNEPQIKQQLDGFRQQIRRHNFPRIGELSFSAGFC
SLAPGAYLPTILDHADKALYYAKEHGRNQVHCYEQLCENGKIASA
QRPFSDDVELFDYKDDDDK
v) VCA0848 (human albumin preproprotein signal
peptide added at N-terminus):
VCA0848 (human albumin preproprotein signal
peptide added at N-terminus) DNA sequence
(codon optimized)-Flag:
(SEQ ID NO: 15)
ATGAAGTGGGTGACCTTTATCTCTCTGCTGTTTCTGTTTTCCTCT
GCCTATTCTATGAACGACAAAGTGCTGGAGTCCGTGATTGAGATT
ACAGAGCAGAAGAACAGCCTGGCCCTGTCCTACTCTATCCTGGCC
ACACTGTCTGAGCTGCTGCCTCTGTCTACAGCCACACTGTTTCAC
CACCTGGGCAGGTCTACCCTGATGGTGGCACGCCTGATCATCACA
AAGAATGCAGCAGGCAAGAAGGAGTATCAGTGGCAGTATGATCAG
GTGTGCGCAGATAATGGATACCAGCACTCTCAGTCTGAGATGGCC
TTTAGCCAGCAGGCCAATGGCCAGTATCAGTGCTTCTGTCCCATC
CCTATTGAGGAGCACTTTTCCGCCGAGCTGTGCCTGATCCTGAAC
AAGGACCCAGAGCCCTACAGGATGCTGATCAACGGCTTCGCCAAG
ATCTACCGCAATTATACCGTGATCCTGCACGAGTCTGAGAGAGAC
AAGCTGACAGGCCTGCTGAATAGAAGAACACTGGAGGATCGGCTG
AGACACACCTTTGCCATCAACCCAAGCACAGAGGAGAATCACAAG
CTGTGGATCGCCATGCTGGACATCGATCACTTCAAGGCCATCAAC
GACCACTTTGGCCACATGATCGGCGATGAGATCCTGCTGATGTTC
GCCCAGCAGATGCAGCACTATTTTGGACCTAGCTCCCAGCTGTTC
CGGTTTGGCGGCGAGGAGTTCGTGATCATCTTTTCTTCTGGAAAT
GAGCCACAGATCAAGCAGCAGCTGGATGGCTTCCGGCAGCAGATC
AGGCGCCACAATTTTCCTAGGATTGGAGAGCTGTCTTTCAGCGCC
GGATTTTGCAGCCTGCGGCCCGGCGATTACCTGCCAACAATCCTG
GACCACGCCGATAAGGCCCTGTACTATGCAAAAGAGCACGGAAGG
AACCAGGTGCACTGCTATGAGCAGCTGTGCGAGAATGGCAAGATC
GCCTCCGCCCAGAGACCATTTTCAGATGATGTGGAACTGTTTGAC
TACAAGGATGATGATGATAAG
VCA0848 (human albumin preproprotein signal
peptide added at N-terminus) protein
sequence-Flag:
(SEQ ID NO: 16)
MKWVTFISLLFLFSSAYSMNDKVLESVIEITEQKNSLALSYSILA
TLSELLPLSTATLFHHLGRSTLMVARLIITKNAAGKKEYQWQYDQ
VCADNGYQHSQSEMAFSQQANGQYQCFCPIPIEEHFSAELCLILN
KDPEPYRMLINGFAKIYRNYTVILHESERDKLTGLLNRRTLEDRL
RHTFAINPSTEENHKLWIAMLDIDHFKAINDHFGHMIGDEILLMF
AQQMQHYFGPSSQLFRFGGEEFVIIFSSGNEPQIKQQLDGFRQQI
RRHNFPRIGELSFSAGFCSLRPGDYLPTILDHADKALYYAKEHGR
NQVHCYEQLCENGKIASAQRPFSDDVELFDYKDDDDK
vi) PA2771 (Dcsbis): PA2771 (Dcsbis)
DNA sequence (codon optimized)-Flag:
(SEQ ID NO: 17)
ATGCTGGCTTGTCCTCTGCCCCCTGACGAAGCTCTGAGACAGCAG
GCTCTGGATGATATGGCTCTGGTGGATACCCCCGCCGAACACTAC
CTGGATGCACTGGTGGAGCTGGCAAGAGAGACATTCGGCGTGAAG
ACAGTGCTGATCTCCCTGATCGACCACGATAGGCAGTGGTTTAAA
GCAAGAATTGGCCTGGATGCAGAGCAGACCCCACGGGATCTGTCC
TTCTGCGGCCACGCCATCCTGGCCTCTGAGCCCCTGATGGTGACC
GACGCCAGCCGGGACCCCCGCTTTCACGATAATCCTCTGGTGACA
GGCCCTCCCTTCATCCGGTTCTACGCAGGAGAGCCACTGCACGCC
TCTAATGGCCAGGCCATCGGCACCCTGTGCCTGATCGACCCAAGC
CCCCGGCTGCTGGATCTGAGAGAGGGCAGACAGCTGAACAGGCTG
AGCATCCTGGCAGAGGGATATCTGCAGCTGAGGTCCCTGACAGAG
CACACAAGGTTTCTGAGACAGGAGATTGATAGAGAGCAGAGGAAG
TCTCTGCTGGACCCCCTGACCCAGCTGTGGAACAGAGCCGGCTTC
CACGCCCTGCACCAGCACGAGCTGGAGCTGGCAAGAGCCTCCGAC
CAGAGAATCGGCATCATCTACTCTGACATCGATCACTTCAAGAGG
ATCAATGACACACTGGGCCACCGGGCCGGCGATTCTGTGCTGAGG
GAGGCAGCAAGCAGACTGAGAGCCGCCCTGAGGCCTGAGGACCTG
CTGGCCCGCTTCGGCGGCGAGGAATTCGTGGCAATGGTGAGGGTG
CGCGAGACAACAGAGCTGACCATGATCGCCAATCGGATCAGAGAG
CTGATGGAGGCAACACCAATCGATTGCGCCGGCACCAGCGTGCCT
GTGACAATCTCCGCCGGCTGTACCCTGGCCGGCTCCGGCGAGGAG
CCAGAGAGGGCCCTGGCCCGCGCCGACGCCGCCCTGTATGATGCC
AAGCGAGCAGGACGGAACCGAGTGGTCTCTGTGGACTACAAGGAC
GATGACGACAAG
PA2771(Dcsbis) protein sequence-Flag:
(SEQ ID NO: 18)
MLACPLPPDEALRQQALDDMALVDTPAEHYLDALVELARETFGVK
TVLISLIDHDRQWFKARIGLDAEQTPRDLSFCGHAILASEPLMVT
DASRDPRFHDNPLVTGPPFIRFYAGEPLHASNGQAIGTLCLIDPS
PRLLDLREGRQLNRLSILAEGYLOLRSLTEHTRFLRQEIDREQRK
SLLDPLTQLWNRAGFHALHQHELELARASDORIGIIYSDIDHFKR
INDTLGHRAGDSVLREAASRLRAALRPEDLLARFGGEEFVAMVRV
RETTELTMIANRIRELMEATPIDCAGTSVPVTISAGCTLAGSGEE
PERALARADAALYDAKRAGRNRVVSVDYKDDDDK
vii) PA2771 (Dcsbis, E10A,
R97A/D100A, R137A/D140A, R207A/D210A,
R225A/D228A, R233A/D236A, R251A/D254A) DNA
sequence (codon optimized)-Flag:
(SEQ ID NO: 19)
ATGCTGGCTTGCCCTCTGCCCCCTGACGCCGCACTGAGACAGCAG
GCTCTGGATGATATGGCTCTGGTGGATACCCCCGCCGAACACTAC
CTGGATGCACTGGTGGAGCTGGCAAGAGAGACATTCGGCGTGAAG
ACAGTGCTGATCTCCCTGATCGACCACGATAGGCAGTGGTTTAAA
GCAAGAATTGGCCTGGATGCAGAGCAGACCCCAAGAGATCTGTCC
TTCTGTGGACACGCAATCCTGGCATCTGAGCCACTGATGGTGACC
GACGCCAGCCGGGACCCCGCCTTTCACGCAAATCCTCTGGTGACA
GGCCCTCCCTTCATCCGCTTTTACGCAGGAGAGCCACTGCACGCC
TCTAATGGCCAGGCCATCGGCACCCTGTGCCTGATCGATCCATCT
CCCGCCCTGCTGGCCCTGAGGGAGGGCCGCCAGCTGAACCGGCTG
AGCATCCTGGCAGAGGGATATCTGCAGCTGAGATCCCTGACAGAG
CACACACGGTTTCTGAGGCAGGAGATTGACAGGGAGCAGAGAAAG
TCTCTGCTGGACCCCCTGACCCAGCTGTGGAACAGGGCCGGCTTC
CACGCCCTGCACCAGCACGAACTGGAGCTGGCAGCAGCATCCGCC
CAGAGAATTGGAATCATCTACTCTGACATCGATCACTTCAAGGCC
ATCAATGCCACACTGGGACACGCAGCAGGAGCATCTGTGCTGCGG
GAGGCCGCCAGCCGGCTGAGAGCCGCCCTGGCCCCTGAGGCCCTG
CTGGCCAGGTTTGGCGGCGAGGAATTCGTGGCAATGGTGAGGGTG
CGCGAGACAACAGAGCTGACCATGATCGCCAATCGGATCAGAGAG
CTGATGGAGGCAACACCAATCGACTGCGCCGGCACCAGCGTGCCT
GTGACAATCTCCGCCGGCTGTACCCTGGCCGGCTCCGGCGAGGAG
CCAGAGCGGGCCCTGGCCAGAGCCGACGCCGCCCTGTATGATGCA
AAACGAGCAGGACGGAATAGAGTGGTCTCAGTGGACTACAAAGAT
GATGACGACAAG
PA2771 (Dcsbis, E10A, R97A/D100A,
R137A/D140A, R207A/D210A, R225A/D228A,
R233A/D236A, R251A/D254A) Protein
sequence-Flag:
(SEQ ID NO: 20)
MLACPLPPDAALRQQALDDMALVDTPAEHYLDALVELARETFGVK
TVLISLIDHDRQWFKARIGLDAEQTPRDLSFCGHAILASEPLMVT
DASRDPAFHANPLVTGPPFIRFYAGEPLHASNGQAIGTLCLIDPS
PALLALREGRQLNRLSILAEGYLQLRSLTEHTRFLRQEIDREQRK
SLLDPLTQLWNRAGFHALHQHELELAAASAQRIGIIYSDIDHFKA
INATLGHAAGASVLREAASRLRAALAPEALLARFGGEEFVAMVRV
RETTELTMIANRIRELMEATPIDCAGTSVPVTISAGCTLAGSGEE
PERALARADAALYDAKRAGRNRVVSVDYKDDDDK
vii) PA3702 (WspR): PA3702(WspR)
DNA sequence (codon optimized)-Flag:
(SEQ ID NO: 21)
ATGCACAACCCTCACGAATCAAAGACCGACCTGGGGGCTCCTCTG
GACGGGGCTGTCATGGTGCTGCTGGTGGACGATCAGGCAATGATT
GGAGAGGCCGTGCGGAGATCCCTGGCATCTGAGGCCGGCATCGAC
TTCCACTTTTGCTCTGATCCTCAGCAGGCAGTGGCAGTGGCAAAC
CAGATCAAGCCAACCGTGATCCTGCAGGACCTGGTCATGCCTGGA
GTGGATGGCCTGACCCTGCTGGCAGCATATAGAGGAAATCCTGCA
ACAAGAGACATCCCAATCATCGTGCTGTCTACCAAGGAGGAGCCA
ACAGTGAAAAGCGCCGCCTTCGCCGCAGGAGCAAACGACTACCTG
GTGAAGCTGCCTGATGCAATTGAACTGGTGGCACGGATCAGATAC
CACTCTCGGAGCTATATCGCCCTGCAGCAGAGAGATGAGGCCTAT
AGGGCCCTGAGAGAGTCTCAGCAGCAGCTGCTGGAGACAAACCTG
GTGCTGCAGCGCCTGATGAATTCTGATGGCCTGACAGGCCTGTCC
AACAGGCGCCACTTTGATGAGTACCTGGAGATGGAGTGGCGGAGA
TCCCTGAGAGAGCAGTCTCAGCTGTCCCTGCTGATGATTGACGTG
GATTACTTCAAGTCTTATAATGACACCTTTGGCCACGTGGCCGGC
GATGAGGCCCTGAGACAGGTGGCAGGAGCAATCCGGGAGGGCTGT
AGCAGAAGCTCTGACCTGGCAGCAAGGTACGGCGGCGAGGAGTTT
GCAATGGTGCTGCCAGGCACCTCCCCTGGCGGCGCCAGGCTGCTG
GCAGAGAAGGTGAGGAGAACAGTGGAGTCTCTGCAGATCTCTCAC
GACCAGCCCAGACCTGGCAGCCACCTGACCGTGTCCATTGGCGTG
TCTACACTGGTGCCTGGCGGCGGCGGCCAGACCTTCCGGGTGCTG
ATTGAGATGGCAGATCAGGCCCTGTATCAGGCCAAGAACAATGGC
CGCAATCAGGTGGGCCTGATGGAGCAGCCAGTGCCACCCGCCCCA
GCAGGAGACTACAAGGACGATGACGACAAA
PA3702(WspR) protein sequence-Flag:
(SEQ ID NO: 22)
MHNPHESKTDLGAPLDGAVMVLLVDDQAMIGEAVRRSLASEAGID
FHFCSDPQQAVAVANQIKPTVILQDLVMPGVDGLTLLAAYRGNPA
TRDIPIIVLSTKEEPTVKSAAFAAGANDYLVKLPDAIELVARIRY
HSRSYIALQQRDEAYRALRESQQQLLETNLVLQRLMNSDGLTGLS
NRRHFDEYLEMEWRRSLREQSQLSLLMIDVDYFKSYNDTFGHVAG
DEALRQVAGAIREGCSRSSDLAARYGGEEFAMVLPGTSPGGARLL
AEKVRRTVESLQISHDQPRPGSHLTVSIGVSTLVPGGGGQTFRVL
IEMADQALYQAKNNGRNQVGLMEQPVPPAPAGDYKDDDDK
viii) PA3702 (WspR, N-terminal 1-175 replaced
with Saccharomyces cerevisiae GCN4(249-278))
DNA sequence (codon optimized)-Flag:
(SEQ ID NO: 23)
AGAATGAAACAGCTGGAGGACAAGGTGGAGGAACTGCTGAGCAAG
AACTACCATCTGGAGAACGAAGTGGCAAGACTGAAAAAACTGGTG
CTGACAGGCCTGTCTAACCGGAGACACTTTGATGAGTACCTGGAG
ATGGAGTGGAGGCGCAGCCTGAGAGAGCAGTCTCAGCTGAGCCTG
CTGATGATCGACGTGGATTACTTCAAGAGCTATAATGACACATTT
GGCCACGTGGCCGGCGATGAGGCCCTGAGGCAGGTGGCCGGCGCC
ATCAGGGAGGGCTGCTCCCGCAGCTCCGATCTGGCCGCCCGCTAT
GGCGGCGAGGAGTTCGCCATGGTGCTGCCAGGCACCTCTCCTGGC
GGCGCCAGACTGCTGGCAGAGAAGGTGCGGAGAACAGTGGAGTCC
CTGCAGATCTCTCACGACCAGCCCCGGCCTGGCTCCCACCTGACC
GTGAGCATCGGCGTGTCCACACTGGTGCCTGGCGGCGGCGGCCAG
ACCTTTCGGGTGCTGATTGAGATGGCAGATCAGGCCCTGTACCAG
GCCAAGAACAATGGCAGAAACCAGGTGGGCCTGATGGAACAGCCA
GTGCCACCAGCCCCAGCAGGAGACTACAAGGACGACGATGACAAA
PA3702 (WspR, N-terminal 1-175 replaced
with Saccharomyces cerevisiae GCN4(249-278))
Protein sequence-Flag:
(SEQ ID NO: 24)
RMKQLEDKVEELLSKNYHLENEVARLKKLVLTGLSNRRHFDEYLE
MEWRRSLREQSQLSLLMIDVDYFKSYNDTFGHVAGDEALRQVAGA
IREGCSRSSDLAARYGGEEFAMVLPGTSPGGARLLAEKVRRTVES
LQISHDQPRPGSHLTVSIGVSTLVPGGGGQTFRVLIEMADQALYQ
AKNNGRNQVGLMEQPVPPAPAGDYKDDDDK
ix) PA3702 (WspR, N-terminal 1-175 replaced
with human albumin preproprotein signal
peptide and Saccharomyces cerevisiae GCN4
(249-278)) DNA sequence (codon optimized)-Flag:
(SEQ ID NO: 25)
ATGAAGTGGGTGACCTTTATCAGTCTGCTGTTTCTGTTCTCAAGC
GCATATAGTAGGATGAAGCAGCTGGAGGACAAAGTGGAGGAACTG
CTGAGCAAGAACTACCACCTGGAGAATGAGGTGGCAAGACTGAAG
AAGCTGGTGCTGACAGGCCTGTCTAACCGGAGACACTTTGACGAG
TATCTGGAGATGGAGTGGAGGCGCAGCCTGAGGGAGCAGTCTCAG
CTGAGCCTGCTGATGATCGACGTGGATTACTTCAAGTCCTATAAT
GACACATTTGGCCACGTGGCCGGCGATGAGGCCCTGAGGCAGGTG
GCCGGCGCCATCAGGGAGGGCTGCTCCCGCAGCTCCGATCTGGCC
GCCCGCTACGGCGGCGAGGAGTTCGCCATGGTGCTGCCAGGCACC
TCTCCTGGCGGCGCCAGACTGCTGGCAGAGAAGGTGCGGAGAACA
GTGGAGTCCCTGCAGATCTCTCACGACCAGCCCCGGCCTGGCTCT
CACCTGACCGTGAGCATTGGCGTGTCCACACTGGTGCCTGGCGGC
GGCGGCCAGACCTTTCGGGTGCTGATTGAGATGGCAGATCAGGCC
CTGTATCAGGCCAAGAACAATGGCAGAAACCAGGTGGGCCTGATG
GAACAGCCAGTGCCCCCAGCCCCAGCAGGAGATTACAAAGATGAC
GACGATAAA
PA3702 (WspR, N-terminal 1-175 replaced
with human albumin preproprotein signal
peptide and Saccharomyces cerevisiae
GCN4(249-278)) protein sequence-Flag:
(SEQ ID NO: 26)
MKWVTFISLLFLFSSAYSRMKQLEDKVEELLSKNYHLENEVARLK
KLVLTGLSNRRHFDEYLEMEWRRSLREQSQLSLLMIDVDYFKSYN
DTFGHVAGDEALRQVAGAIREGCSRSSDLAARYGGEEFAMVLPGT
SPGGARLLAEKVRRTVESLQISHDQPRPGSHLTVSIGVSTLVPGG
GGQTFRVLIEMADQALYQAKNNGRNQVGLMEQPVPPAPAGDYKDD
DDK
x) ECSP_2022(ydeH)-Flag:
ECSP_2022(ydeH) DNA sequence
(codon optimized)-Flag:
(SEQ ID NO: 27)
ATGATCAAGAAGACTACCGAGATTGACGCTATCCTGCTGAACCTG
AATAAGGCTATCGACGCACATTACCAGTGGCTGGTGTCCATGTTC
CACTCTGTGGTGGCAAGGGATGCATCCAAGCCTGAGATCACCGAT
AACCACTCCTACGGCCTGTGCCAGTTTGGCCGGTGGATTGACCAC
CTGCGGCCCCTGGACAATGATGAGCTGCCTTATGTGCGGCTGATG
GATTCTGCCCACCAGCACATGCACAACTGTGGGAGGGAGCTGATG
CTGGCAATTGTGGAGAATCACTGGCAGGATGCACACTTTGATGCA
TTTCAGGAGGGCGTGCTGTCCTTCACCGCCGCCCTGACAGACTAC
AAGATCTATCTGCTGACCATCCGGTCTAACATGGATGTGCTGACA
GGCCTGCCAGGCCGGAGAGTGCTGGACGAGAGCTTCGATCACCAG
CTGAGGAACACAGAGCCCCTGAATCTGTACCTGATGCTGCTGGAC
ATCGATCGCTTTAAGCTGGTGAATGACACATATGGACACCTGATT
GGCGATGTGGTGCTGAGAACCCTGGCAACATACCTGGCATCCTGG
ACCAGGGACTATGAGACAGTGTACAGATATGGCGGCGAGGAGTTC
ATCATCATCGTGAAGGCCGCCAATGATGAGGAGGCCTGCAGGGCA
GGCGTGCGCATCTGTCAGCTGGTGGATAATCACGCAATCACCCAC
TCTGAGGGACACATCAATATCACCGTGACAGCAGGCGTGAGCAGG
GCATTCCCTGAAGAGACACTGGACGTGGTCATCGGAAGAGCCGAT
AGAGCAATGTATGAGGGCAAGCAGACAGGAAGGAATCGCTGCATG
TTTATTGACGAGCAGAATGTGATTAACCGGGTGGACTACAAAGAC
GACGATGACAAG
ECSP_2022(ydeH) protein sequence-Flag:
(SEQ ID NO: 28)
MIKKTTEIDAILLNLNKAIDAHYQWLVSMFHSVVARDASKPEITD
NHSYGLCQFGRWIDHLRPLDNDELPYVRLMDSAHQHMHNCGRELM
LAIVENHWQDAHFDAFQEGVLSFTAALTDYKIYLLTIRSNMDVLT
GLPGRRVLDESFDHQLRNTEPLNLYLMLLDIDRFKLVNDTYGHLI
GDVVLRTLATYLASWTRDYETVYRYGGEEFIIIVKAANDEEACRA
GVRICQLVDNHAITHSEGHINITVTAGVSRAFPEETLDVVIGRAD
RAMYEGKQTGRNRCMFIDEQNVINRVDYKDDDDK
xi) ECSP_2022(ydeH, R56A/D59A, R62A/D65A,
R73A/D76A, R141A/D144A) DNA sequence
(codon optimized)-Flag:
(SEQ ID NO: 29)
ATGATTAAGAAAACAACAGAGATTGACGCTATCCTGCTGAACCTG
AACAAGGCTATCGACGCACATTACCAGTGGCTGGTGTCTATGTTC
CACTCTGTGGTGGCAAGAGATGCATCCAAGCCTGAGATCACCGAT
AACCACTCCTACGGCCTGTGCCAGTTTGGAGCATGGATTGCACAC
CTGGCCCCTCTGGCCAATGATGAACTGCCTTATGTGGCCCTGATG
GCATCTGCCCACCAGCACATGCACAACTGTGGAAGGGAGCTGATG
CTGGCAATTGTGGAGAATCACTGGCAGGATGCACACTTTGATGCA
TTTCAGGAGGGCGTGCTGTCCTTCACCGCCGCCCTGACAGACTAC
AAGATCTATCTGCTGACCATCCGGTCTAATATGGATGTGCTGACA
GGCCTGCCAGGAAGAGCCGTGCTGGCAGAGAGCTTCGACCACCAG
CTGAGGAACACAGAGCCCCTGAATCTGTACCTGATGCTGCTGGAC
ATCGATCGCTTTAAGCTGGTGAATGACACATATGGCCACCTGATT
GGCGATGTGGTGCTGCGGACCCTGGCAACATACCTGGCCTCCTGG
ACCAGGGATTATGAGACAGTGTACAGATATGGCGGCGAGGAGTTC
ATCATCATCGTGAAAGCAGCAAATGATGAGGAGGCCTGCAGAGCC
GGCGTGAGAATCTGTCAGCTGGTGGATAATCACGCAATCACCCAC
TCTGAGGGACACATCAATATCACCGTGACAGCAGGCGTGAGCAGA
GCATTTCCTGAGGAGACACTGGACGTGGTCATCGGAAGGGCAGAT
AGAGCAATGTATGAGGGCAAGCAGACAGGACGGAATAGATGCATG
TTTATTGACGAGCAGAATGTGATTAACCGGGTGGATTACAAAGAC
GACGACGACAAA
ECSP_2022(ydeH, R56A/D59A, R62A/D65A,
R73A/D76A, R141A/D144A) protein sequence-Flag:
(SEQ ID NO: 30)
MIKKTTEIDAILLNLNKAIDAHYQWLVSMFHSVVARDASKPEITD
NHSYGLCQFGAWIAHLAPLANDELPYVALMASAHQHMHNCGRELM
LAIVENHWQDAHFDAFQEGVLSFTAALTDYKIYLLTIRSNMDVLT
GLPGRAVLAESFDHQLRNTEPLNLYLMLLDIDRFKLVNDTYGHLI
GDVVLRTLATYLASWTRDYETVYRYGGEEFIIIVKAANDEEACRA
GVRICQLVDNHAITHSEGHINITVTAGVSRAFPEETLDVVIGRAD
RAMYEGKQTGRNRCMFIDEQNVINRVDYKDDDDK
xii) CdaA:
CdaA DNA sequence (codon optimized)-Flag:
(SEQ ID NO: 31)
ATGGACTTCAGCAACATGAGCATCCTGCACTATCTGGCTAATATC
GTGGACATTCTGGTCGTCTGGTTCGTGATCTACAAGGTCATCATG
CTGATCAGAGGAACAAAGGCAGTGCAGCTGCTGAAGGGCATCTTC
ATCATCATTGCAGTGAAGCTGCTGTCTGGCTTCTTTGGCCTGCAG
ACAGTGGAGTGGATCACAGATCAGATGCTGACCTGGGGCTTCCTG
GCAATCATCATCATCTTTCAGCCTGAGCTGAGAAGAGCCCTGGAG
ACACTGGGAAGAGGCAACATCTTTACCAGATATGGCAGCAGGATT
GAGAGAGAGCAGCACCACCTGATCGAGAGCATCGAGAAGTCCACC
CAGTATATGGCCAAAAGAAGAATTGGCGCCCTGATTTCTGTGGCA
AGGGATACAGGAATGGACGATTACATCGAGACAGGCATCCCACTG
AACGCCAAGATCAGCTCCCAGCTGCTGATCAACATCTTCATCCCT
AATACACCTCTGCACGATGGAGCCGTGATCATCAAGGGAAATGAG
ATTGCATCCGCCGCATCTTATCTGCCACTGTCTGATTCTCCCTTT
CTGTCTAAGGAACTGGGAACCAGACACAGAGCCGCCCTGGGCATC
TCTGAGGTGACCGACAGCATTACCATTGTGGTGTCCGAGGAGACA
GGCGGCATCTCTCTGACCAAGGGCGGCGAGCTGTTCCGCGACGTG
AGCGAGGAGGAGCTGCACAAGATCCTGCTGAAGGAGCTGGTGACA
GTGACAGCCAAGAAGCCCAGCATTTTCTCAAAGTGGAAGGGCGGG
AAGAGCGAGGACTACAAAGACGATGATGATAAG
CdaA protein sequence-Flag:
(SEQ ID NO: 32)
MDFSNMSILHYLANIVDILVVWFVIYKVIMLIRGTKAVOLLKGIF
IIIAVKLLSGFFGLQTVEWITDQMLTWGFLAIIIIFQPELRRALE
TLGRGNIFTRYGSRIEREQHHLIESIEKSTQYMAKRRIGALISVA
RDTGMDDYIETGIPLNAKISSQLLINIFIPNTPLHDGAVIIKGNE
IASAASYLPLSDSPFLSKELGTRHRAALGISEVTDSITIVVSEET
GGISLTKGGELFRDVSEEELHKILLKELVTVTAKKPSIFSKWKGG
KSEDYKDDDDK
xiii) CdaA (N-terminal 1-80 deletion)
DNA sequence (codon optimized)-Flag:
(SEQ ID NO: 33)
TTTCAGCCCGAACTGCGGAGAGCACTGGAGACCCTGGGGAGAGGA
AATATCTTCACTCGCTATGGGAGCCGGATTGAGAGAGAACAGCAC
CACCTGATCGAGAGCATCGAGAAGTCCACCCAGTACATGGCAAAG
AGAAGAATTGGCGCCCTGATCTCTGTGGCAAGAGACACCGGCATG
GACGATTATATCGAGACAGGCATCCCTCTGAACGCCAAGATCAGC
TCCCAGCTGCTGATCAACATCTTCATCCCTAATACCCCTCTGCAC
GATGGAGCCGTGATCATCAAGGGAAATGAGATTGCATCCGCCGCA
TCTTACCTGCCACTGTCTGATAGCCCCTTTCTGAGCAAGGAACTG
GGAACAAGGCACAGAGCCGCCCTGGGCATCTCTGAGGTGACCGAC
AGCATTACCATTGTGGTGTCCGAGGAGACAGGCGGCATCTCTCTG
ACAAAGGGCGGCGAGCTGTTCAGGGACGTGAGCGAGGAGGAGCTG
CACAAGATCCTGCTGAAGGAGCTGGTGACAGTGACAGCCAAGAAG
CCCTCAATCTTCAGCAAGTGGAAAGGAGGGAAAAGCGAGGACTAC
AAGGACGACGACGATAAG
CdaA (N-terminal 1-80 deletion)
protein sequence-Flag:
(SEQ ID NO: 34)
FQPELRRALETLGRGNIFTRYGSRIEREQHHLIESIEKSTQYMAK
RRIGALISVARDTGMDDYIETGIPLNAKISSQLLINIFIPNTPLH
DGAVIIKGNEIASAASYLPLSDSPFLSKELGTRHRAALGISEVTD
SITIVVSEETGGISLTKGGELFRDVSEEELHKILLKELVTVTAKK
PSIFSKWKGGKSEDYKDDDDK

All relevant portions of references referred to herein are incorporated by reference.

1. A method of constitutively expressing a bacterial dinucleotide cyclase or a functional domain thereof in a mammalian cell comprising transfecting the mammalian cell with a transgene encoding the bacterial dinucleotide cyclase and subjecting the mammalian cell to suitable growth conditions.

2. The method of claim 1, wherein the mammalian cell is an immune cell selected from the group consisting of neutrophils, eosinophils, basophils, mast cells, monocytes, macrophages, dendritic cells, natural killer cells, and lymphocytes.

3. The method of claim 1, wherein the mammalian cell is a cancer or tumour cell.

4. The method of claim 1, wherein the bacterial dinucleotide cyclase catalyzes the synthesis of a cyclic dinucleotide selected from the group consisting of: c-di-GMP, c-di-AMP and cGAMP.

5. The method of claim 1, wherein the bacterial dinucleotide cyclase is from Vibrio cholera, Listeria monocytogenes or Mycobacterium tuberculosis.

6. The method of claim 1, wherein the functional domain comprises the catalytic domain of the bacterial dinucleotide cyclase.

7. The method of claim 1, wherein the transgene is incorporated into a plasmid, cosmid or viral vector.

8. The method of claim 7, wherein the viral vector is replication-competent.

9. The method of claim 7, wherein the viral vector is a DNA virus or RNA virus.

10. The method of claim 7, wherein the viral vector is an oncolytic virus.

11. A method of expressing a bacterial dinucleotide cyclase in a tumour or cancer cell comprising introducing a transgene encoding the dinucleotide cyclase into the cell.

12. The method of claim 11, wherein the transgene is incorporated into a viral vector.

13. The method of claim 12, wherein the viral vector is replication-competent.

14. The method of claim 12, wherein the viral vector is an oncolytic virus.

15. A method of treating cancer in a mammal comprising administering a vector expressing a dinucleotide cyclase to the mammal.

16. The method of claim 15, wherein the vector is a replication-competent viral vector.

17. The method of claim 15, wherein the vector is an oncolytic virus.

18. The method of claim 15, wherein the vector is administered intravenously, intramuscularly, intratumorally, or intranasally.

19. The method of claim 15, wherein the vector is administered with an immunotherapy drug.

20. An oncolytic viral vector expressibly incorporating a transgene encoding a bacterial dinucleotide cyclase.
